(19) United States
(12) Patent Application Publication
(10) Pub. No.: US 2017/0037030 A1
(43) Pub. Date: Feb. 9, 2017

(54) AUTOTAXIN INHIBITORS

(71) Applicants: Vikki FURMINGER, Seaford Sussex (GB); Owen HUGHES, Repton Derbyshire (GB); Darren LEGRAND, East Grinstead Sussex (GB); Emily STANLEY, Horsham (GB); Christopher THOMSON, Cambridge, MA (US); Gareth WILLIAMS, Horsham (GB)

(72) Inventors: Vikki Furminger, Seaford (GB); Owen Rhys Hughes, Nottingham (GB); Darren Mark Legrand, East Grinstead (GB); Emily Stanley, Horsham (GB); Christopher Thomson, Cambridge, MA (US); Gareth Williams, Horsham (GB)

(73) Assignee: NOVARTIS AG, Basel (CH)

(21) Appl. No.: 15/305,602

(22) PCT Filed: Apr. 21, 2015
(86) PCT No.: PCT/IB2015/052912

§ 371 Date: Oct. 20, 2016

(30) Foreign Application Priority Data
Apr. 24, 2014 (EP) 14165810.4

(51) Int. Cl. C07D 401/12; A61K 31/454

(52) U.S. Cl. CPC C07D 401/12; A61K 31/454

(57) ABSTRACT

The present invention relates to novel compounds that are autotaxin inhibitors, processes for their preparation, pharmaceutical compositions and medicaments containing them and to their use in the treatment of an ATX-dependent or ATX-mediated disease or condition.

### TECHNICAL FIELD

The present invention relates to novel compounds that are autotaxin inhibitors, processes for their preparation, pharmaceutical compositions and medicaments containing them and to their use in diseases and disorders mediated by autotaxin.

### BACKGROUND

Autotaxin (ATX), also known as ectonucleotide pyrophosphatase/phosphodiesterase (ENPP2), is a secreted ectoenzyme known to possess lysophospholipase D activity (Umezu-Goto et al., 2002), and is responsible for producing the bioactive lipid mediator lysophosphatidic acid (LPA) by the hydrolysis of lysophosphatidylcholine (LPC) (Tokumura et al., 2002). LPA is highly implicated in the pathogenesis of a number of physio-pathological diseases, including cancer (Liu et al., 2009; Mills & Moolenaar, 2003), neuropathic pain (Inoue et al., 2004) and fibrosis (Tager et al., 2008). Following the production of LPA, the lipid binds to specific G protein-coupled receptors of which there are seven known isoforms (Noguchi et al., 2009). Binding of LPA activates multiple signalling pathways (Mills & Moolenaar, 2003) including cell migration (van Dijk et al., 1998), proliferation and survival (Brindley, 2004). Other cellular responses include smooth muscle contraction, apoptosis and platelet aggregation (Tigyi & Parrill, 2003).

ATX was originally identified as a cell motility-stimulating factor following isolation from human A2058 melanoma cells (Stracke et al., 1992). Subsequent work on the enzyme was focused towards its role as a motility factor due to its aberrant expression in many cancer types including breast and renal cancer (Stassar et al., 2001), Hodgkin's lymphoma (Baumforth et al., 2005), follicular lymphoma (Masuda et al., 2008), as well as fibrosis of the lung and kidney (Hama et al., 2004). Ten years following its discovery, ATX was characterised as a secreted lysophospholipase (lysoPLD) (Tokumura et al., 2002; Gesta et al., 2002). Since then ATX gene knockout mice have shown that the ATX-LPA signalling axis plays a vital role during embryonic development of the cardiovascular and neural system (Tanaka et al., 2006; van Meeteren et al., 2006), resulting in early embryonic lethality (Bachner et al., 1999).

ATX belongs to a family of proteins called nucleotide pyrophosphatase/phosphodiesterase (NPP), encoded for by the gene ENPP. The family consists of seven structurally related enzymes (ENPP 1-7) conserved within vertebrates which are numbered according to their discovery. They were originally defined by their ability to hydrolyse pyrophosphate or phosphodiester bonds of various nucleotides and nucleotides derivatives in vitro (Stefan et al., 1999; Goding et al., 1998; Gijsbers et al., 2001), though ENPP2 and choline phosphate esters (ENPP6 & 7) have specific activity for other extracellular non-nucleotide molecules. ENPP2 (ATX) is unique within the family as it is the only secreted protein, whereas other ENPP members are transmembrane proteins (Stefan et al., 2005).

WO02/100352 (Merck) and WO 02/080928 (Merck) relate to N-substituted nonaryl-heterocyclo amidyl NMDA/NR2B receptor antagonists for the treatment or prevention of migraines.

WO2010/115491 (Merck) and WO 2009/046841 (Merck) relate to piperidine and piperazine derivatives as ATX inhibitors.

WO2010/112116 (Merck) and WO2010/112124 (Merck) relate to heterocyclic compounds as ATX inhibitors and WO 2011/044978 (Merck) relates to sulfoxide derivatives for treating tumours.

Hence, there is a need for further potent inhibitors of ATX.

### SUMMARY OF THE INVENTION

In a first aspect, the invention relates to a compound of formula (I)

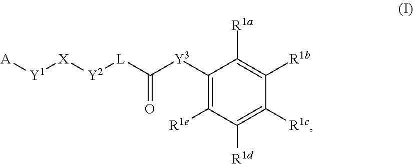

or a pharmaceutically acceptable salt thereof, wherein
A is selected from

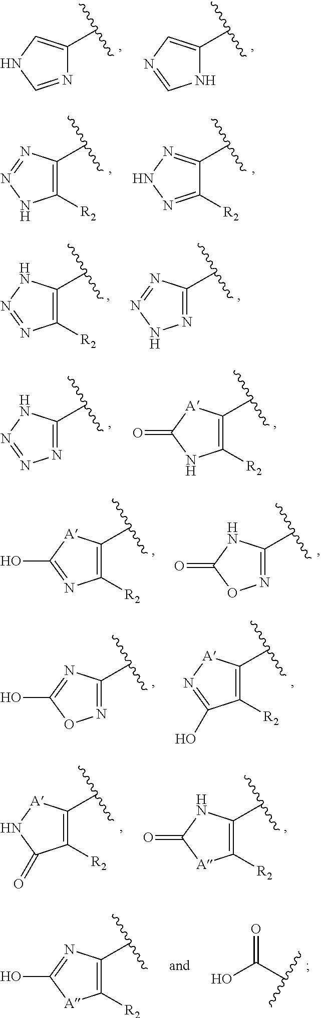

A′ is selected from O, S and NR^{2a};
A″ is selected from O and S;
Y¹ is —C(═O)—(CR^{2b}R^{2c})ₘ— or —C(OH)—(CR^{2b}R^{2c})ₘ—;
X is selected from —C(═O)—, —N(R³)—C(═O)—, C(═O)—N(R³)—, —N(R³)— and CH₂—;
Y² is —(CR^{4a}R^{4b})ₙ—;
m is selected from 0, 1, 2, 3 and 4;
n is selected from 0, 1, 2, 3, 4 and 5;
wherein when Y¹ is —(CR^{2b}R^{2c})ₙ— and A is not HO—C(═O)—, the sum of m and n is not less than 2 and no more than 5; and
wherein when Y¹ is —(CR^{2b}R^{2c})ₘ— and A is HO—C(═O)—, the sum of m and n is not less than 2 and no more than 7; or

### A-Y¹—X— is

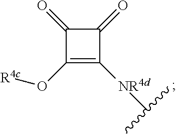

L is selected from

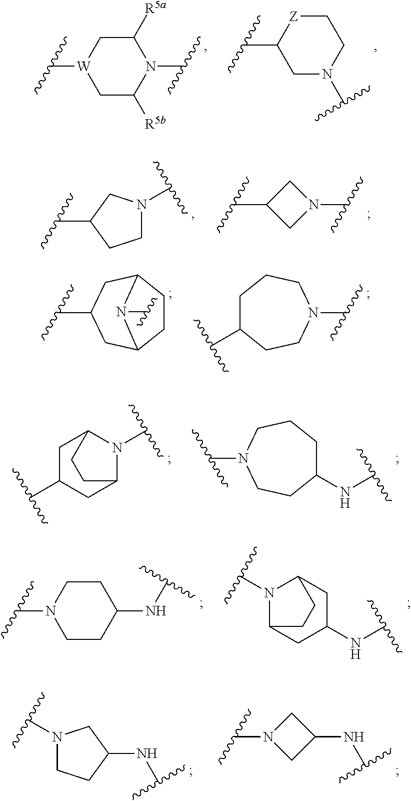

### W is CH or N;

Z is selected from CH₂, O and NR^{5c};
Y³ is selected from —O—(CR^{6a}R^{6b})—, —(CR^{6c}R^{6d})—O—, —CH═CH—, —CR^{6e}R^{6f}—CR^{6g}R^{6h}—, and —O—(CR⁶ⁱR^{6j}—CR^{6k}R^{6l})—;
R^{1a}, R^{1b}, R^{1c}, R^{1d} and R^{1e} are defined according to any one of
(a) R^{1b} is halogen; R^{1d} is halogen, CN, C₄alkyl, C₁₋₄haloalkyl or C₁₋₄haloalkoxy; and R^{1a}, R^{1c} and R^{1e} are H;
(b) R^{1b} is halogen; R^{1d} is halogen, CN, C₄alkyl, C₁₋₄haloalkyl or C₁₋₄haloalkoxy; R^{1c} is halogen; and R^{1a} and R^{1e} are H;
(c) R^{1b} is C₁₋₄alkyl; R^{1d} is C₁₋₄alkyl, C₄haloalkyl, C₁₋₄haloalkoxy or CN; R^{1a}, R^{1c} and R^{1e} are H;
(d) R^{1b} is CN; R^{1d} is C₁₋₄haloalkyl or C₁₋₄haloalkoxy; and R^{1a}, R^{1c} and R^{1e} are H;
(e) R^{1b} is C₁₋₄haloalkyl or C₁₋₄haloalkoxy; and R^{1a}, R^{1c} and R^{1e} are H; and R^{1d} is H or CN;
(f) R^{1a} is halogen; R^{1c} is halogen, CN, C₄alkyl, C₁₋₄haloalkyl or C₁₋₄haloalkoxy; and R^{1b}, R^{1d} and R^{1e} are H;
(g) R^{1c} is halogen, CN, C₄alkyl, C₁₋₄haloalkyl or C₁₋₄haloalkoxy; and R^{1a}, R^{1b} and R^{1e} are H; and R^{1d} is halogen, CN, C₄alkyl, C₄haloalkyl, C₁₋₄haloalkoxy, or H;
R² is selected from H, C₁₋₄alkyl and halogen;
R^{2a}, R^{2b}, R^{2c}, R³, R^{4a}, R^{4b}, R^{4c}, R^{4d}, R^{5a}, R^{5b}, R^{5c}, R^{6a}, R^{6b}, R^{6c}, R^{6d}, R^{6e}, R^{6f}, R^{6g}, R^{6h}, R⁶ⁱ, R^{6j}, R^{6k} and R^{6l} are independently selected from H and C₁₋₄alkyl.

In other aspects, the invention relates to pharmaceutical compositions and combinations comprising compounds of the first aspect, and to the use of such compounds of the first aspect in the treatment of an ATX-dependent or ATX-mediated disease or condition.

### DESCRIPTION OF THE EMBODIMENTS

In embodiment 1 of the invention, there is provided a compound of formula (I)

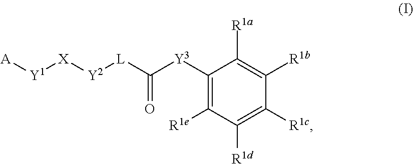

or a pharmaceutically acceptable salt thereof, wherein
A is selected from

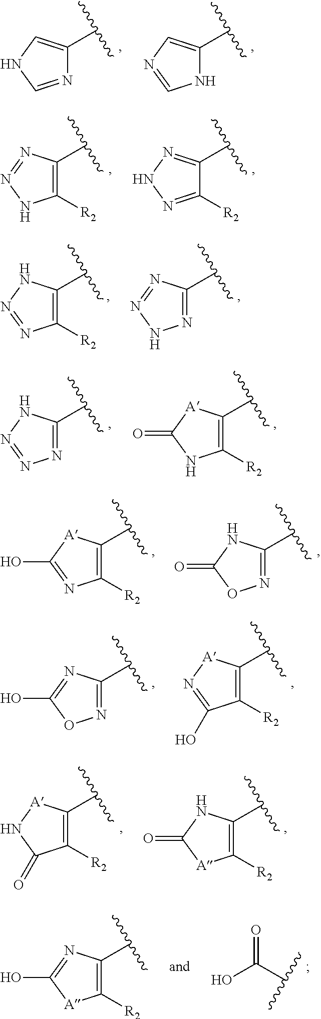

A′ is selected from O, S and NR^{2a};
A″ is selected from O and S;
Y¹ is —C(═O)—(CR^{2b}R^{2c})ₘ— or —C(OH)—(CR^{2b}R^{2c})ₘ—;
X is selected from —C(═O)—, —N(R³)—C(═O)—, C(═O)—N(R³)—, —N(R³)— and CH₂—;
Y² is —(CR^{4a}R^{4b})ₙ—;
m is selected from 0, 1, 2, 3 and 4;
n is selected from 0, 1, 2, 3, 4 and 5;
wherein when Y¹ is —(CR^{2b}R^{2c})ₘ— and A is not HO—C(═O)—, the sum of m and n is not less than 2 and no more than 5; and
wherein when Y¹ is —(CR^{2b}R^{2c})ₘ— and A is HO—C(═O)—, the sum of m and n is not less than 2 and no more than 7; or

### A-Y¹—X— is

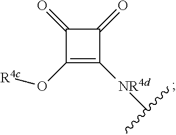

L is selected from

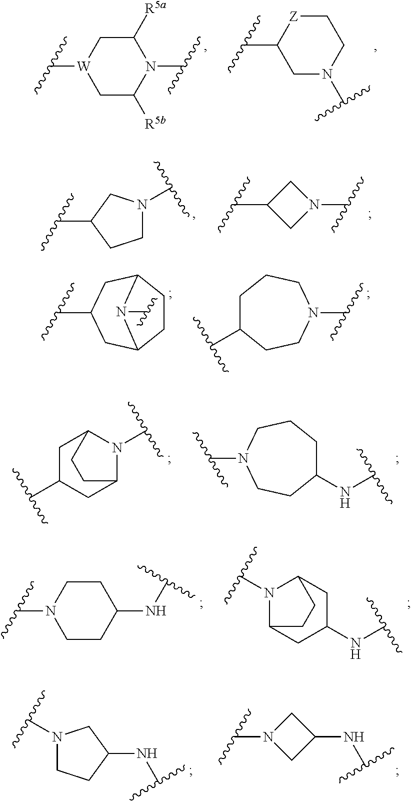

### W is CH or N;

Z is selected from CH₂, O and NR^{5c};
Y³ is selected from —O—(CR^{6a}R^{6b})—, —(CR^{6c}R^{6d})—O—, —CH═CH—, —CR^{6e}R^{6f}—CR^{6g}R^{6h}—, and —O—(CR⁶ⁱR^{6j}—CR^{6k}R^{6l})—;
R^{1a}, R^{1b}, R^{1c}, R^{1d} and R^{1e} are defined according to any one of
(a) R^{1b} is halogen; R^{1d} is halogen, CN, C₄alkyl, C₁₋₄haloalkyl or C₁₋₄haloalkoxy; and R^{1a}, R^{1c} and R^{1e} are H;
(b) R^{1b} is halogen; R^{1d} is halogen, CN, C₄alkyl, C₁₋₄haloalkyl or C₁₋₄haloalkoxy; R^{1c} is halogen; and R^{1a} and R^{1e} are H;
(c) R^{1b} is C₁₋₄alkyl; R^{1d} is C₁₋₄alkyl, C₄haloalkyl, C₁₋₄haloalkoxy or CN; R^{1a}, R^{1c} and R^{1e} are H;
(d) R^{1b} is CN; R^{1d} is C₁₋₄haloalkyl or C₁₋₄haloalkoxy; and R^{1a}, R^{1c} and R^{1e} are H;
(e) R^{1b} is C₁₋₄haloalkyl or C₁₋₄haloalkoxy; and R^{1a}, R^{1c} and R^{1e} are H; and R^{1d} is H or CN;
(f) R^{1a} is halogen; R^{1c} is halogen, CN, C₄alkyl, C₁₋₄haloalkyl or C₁₋₄haloalkoxy; and R^{1b}, R^{1d} and R^{1e} are H;
(g) R^{1c} is halogen, CN, C₄alkyl, C₁₋₄haloalkyl or C₁₋₄haloalkoxy; and R^{1a}, R^{1b} and R^{1e} are H;
and R^{1d} is halogen, CN, C₄alkyl, C₄haloalkyl, C₁₋₄haloalkoxy, or H;
R² is selected from H, C₁₋₄alkyl and halogen;
R^{2a}, R^{2b}, R^{2c}, R³, R^{4a}, R^{4b}, R^{4c}, R^{4d}, R^{5a}, R^{5b}, R^{5c}, R^{6a}, R^{6b}, R^{6c}, R^{6d}, R^{6e}, R^{6f}, R^{6g}, R^{6h}, R⁶ⁱ, R^{6j}, R^{6k} and R^{6l} are independently selected from H and C₁₋₄alkyl.

In embodiment 1.1 of the invention, there is provided a compound of formula (I)

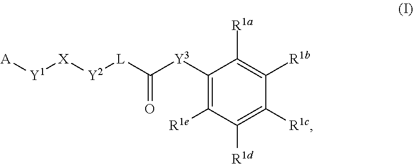

or a pharmaceutically acceptable salt thereof, wherein
A is selected from

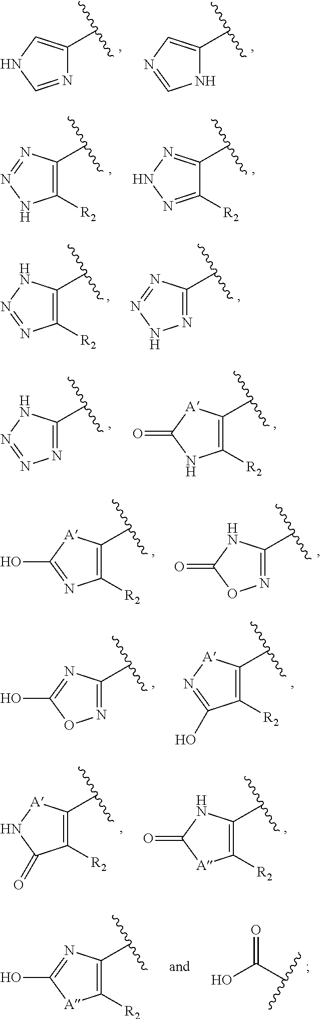

A′ is selected from O, S and NR^{2a};
A″ is selected from O and S;
Y¹ is —C(═O)—(CR^{2b}R^{2c})ₘ— or —C(OH)—(CR^{2b}R^{2c})ₘ—;
X is selected from —C(═O)—, —N(R³)—C(═O)— and —C(═O)—N(R³)—;
Y² is —(CR^{4a}R^{4b})ₙ—;
m is selected from 0, 1, 2, 3 and 4;
n is selected from 0, 1, 2, 3, 4 and 5;
wherein when Y¹ is —(CR^{2b}R^{2c})ₙ— and A is not HO—C(═O)—, the sum of m and n is not less than 2 and no more than 5; and
wherein when Y¹ is —(CR^{2b}R^{2c})ₙ— and A is HO—C(═O)—, the sum of m and n is not less than 2 and no more than 7; or

### A-Y¹—X— is

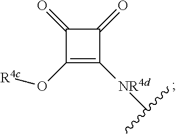

L is selected from

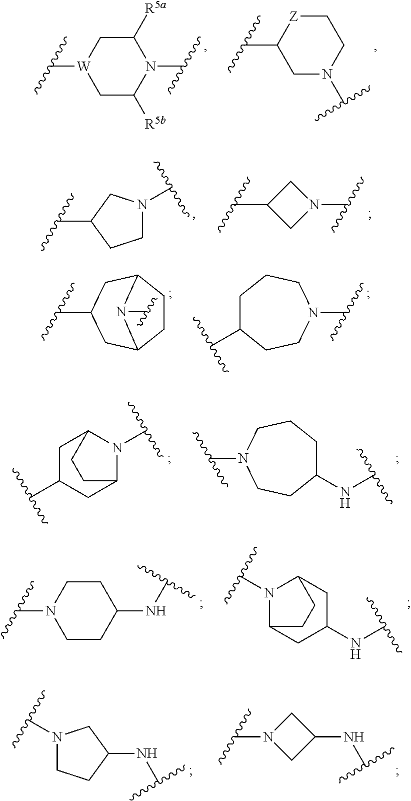

### W is CH or N;

Z is selected from CH₂, O and NR^{5c};
Y³ is selected from —O—(CR^{6a}R^{6b})—, —(CR^{6c}R^{6d})—O—, —CH═CH—, —CR^{6e}R^{6f}—CR^{6g}R^{6h}—, and —O—(CR⁶ⁱR^{6j}—CR^{6k}R^{6l})—;
R^{1a}, R^{1b}, R^{1c}, R^{1d} and R^{1e} are defined according to any one of
(a) R^{1b} is halogen; R^{1d} is halogen, CN, C₁₋₄alkyl, C₁₋₄haloalkyl or C₁₋₄haloalkoxy; and R^{1a}, R^{1c} and R^{1e} are H;
(b) R^{1b} is halogen; R^{1d} is halogen, CN, C₄alkyl, C₁₋₄haloalkyl or C₁₋₄haloalkoxy; R^{1c} is halogen; and R^{1a} and R^{1e} are H;
(c) R^{1b} is C₁₋₄alkyl; R^{1d} is C₁₋₄alkyl, C₄haloalkyl, C₁₋₄haloalkoxy or CN; R^{1a}, R^{1c} and R^{1e} are H;
(d) R^{1b} is CN; R^{1d} is C₁₋₄haloalkyl or C₁₋₄haloalkoxy; and R^{1a}, R^{1c} and R^{1e} are H;
(e) R^{1b} is C₁₋₄haloalkyl or C₁₋₄haloalkoxy; and R^{1a}, R^{1c} and R^{1e} are H; and R^{1d} is H or CN;
(f) R^{1a} is halogen; R^{1c} is halogen, CN, C₄alkyl, C₁₋₄haloalkyl or C₁₋₄haloalkoxy; and R^{1b}, R^{1d} and R^{1e} are H;
(g) R^{1c} is halogen, CN, C₄alkyl, C₁₋₄haloalkyl or C₁₋₄haloalkoxy; and R^{1a}, R^{1b} and R^{1e} are H;
and R^{1d} is halogen, CN, C₄alkyl, C₄haloalkyl, C₁₋₄haloalkoxy, or H;
R² is selected from H, C₁₋₄alkyl and halogen;
R^{2a}, R^{2b}, R^{2c}, R³, R^{4a}, R^{4b}, R^{4c}, R^{4d}, R^{5a}, R^{5b}, R^{5c}, R^{6a}, R^{6b}, R^{6c}, R^{6d}, R^{6e}, R^{6f}, R^{6g}, R^{6h}, R⁶ⁱ, R^{6j}, R^{6k} and R^{6l} are independently selected from H and C₁₋₄alkyl.

In embodiment 1.2 of the invention, there is provided a compound of formula (I)

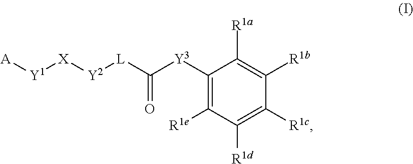

or a pharmaceutically acceptable salt thereof, wherein
A is selected from

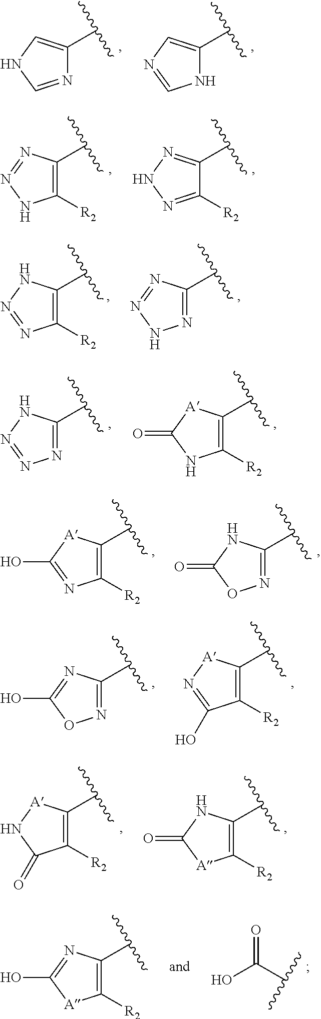

A′ is selected from O, S and NR^{2a};
A″ is selected from O and S;
Y¹ is —C(═O)—(CR^{2b}R^{2c})ₘ— or —C(OH)—(CR^{2b}R^{2c})ₘ—;
X is selected from —C(═O)—, —N(R³)—C(═O)— and —C(═O)—N(R³)—;
Y² is —(CR^{4a}R^{4b})ₙ—;
m is selected from 0, 1, 2, 3 and 4;
n is selected from 0, 1, 2, 3, 4 and 5;
wherein when Y¹ is —(CR^{2b}R^{2c})ₙ— the sum of m and n is not less than 2 and no more than 5;
or

### A-Y¹—X— is

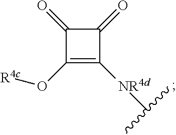

L is selected from

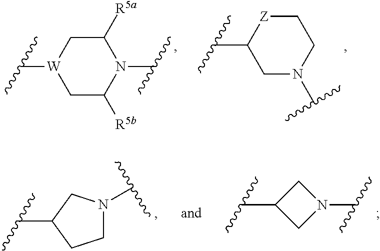

### W is CH or N;

Z is selected from CH₂, O and NR^{5c};
Y³ is selected from —O—(CR^{6a}R^{6b})—, (CR^{6c}R^{6d})—O—, —CH═CH— and CR^{6e}R^{6f}—CR^{6g}R^{6h}—;
R^{1a}, R^{1b}, R^{1c}, R^{1d} and R^{1e} are defined according to any one of
(a) R^{1b} and R^{1d} is halogen, and R^{1a}, R^{1c} and R^{1e} is H;
(b) R^{1a} and R^{1c} is halogen, and R^{1b}, R^{1d} and R^{1e} is H;
(c) R^{1c} is C₁₋₄haloalkyl, in particular CF₃, or C₁₋₄haloalkoxy, and R^{1a}, Rib and R^{1e} are H, and R^{1d} is halogen, C₁₋₄alkyl, particularly methyl, or H;
(d) R^{1b} is C₁₋₄haloalkyl, in particular CF₃, or C₁₋₄haloalkoxy, and R^{1a}, R^{1c} and R^{1e} are H, and R^{1d} is halogen, C₁₋₄alkyl, particularly methyl, or H;
(e) R^{1b} is C₁₋₄alkyl, R^{1d} is halogen, and R^{1a}, R^{1c} and R^{1e} is H; and
(f) R^{1b} is CN, R^{1d} is halogen, and R^{1a}, R^{1c} and R^{1e} is H;
R² is selected from H, C₁₋₄alkyl and halogen;
R^{2a}, R^{2b}, R^{2C}, R³, R^{4a}, R^{4b}, R^{4c}, R^{4d}, R^{5a}, R^{5b}, R^{5c}, R^{6a}, R^{6b}, R^{6c}, R^{6d}, R^{6e}, R^{6f}, R^{6g} and R^{6l} are independently selected from H and C₁₋₄alkyl.

In embodiment 2 of the invention, there is provided a compound of formula (I)

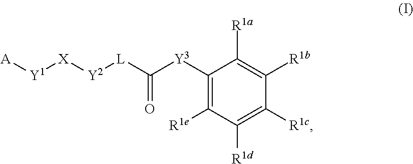

or a pharmaceutically acceptable salt thereof, wherein
A is selected from

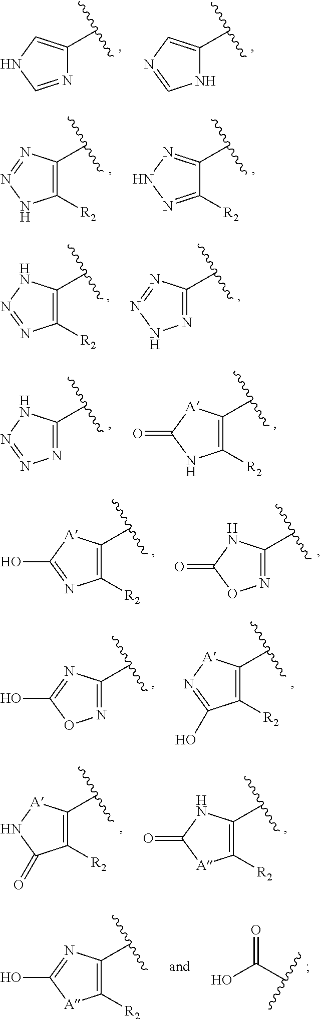

A′ is selected from O, S and NR^{2a};
A″ is selected from O and S;
Y¹ is —C(═O)—(CR^{2b}R^{2c})ₙ— or —C(OH)—(CR^{2b}R^{2c})ₘ—;
X is selected from —C(═O)—, —N(R³)—C(═O)— and —C(═O)—N(R³)—;
Y² is —(CR^{4a}R^{4b})ₙ—;
m is selected from 0, 1, 2, 3 and 4;
n is selected from 0, 1, 2, 3, 4 and 5;
wherein when Y¹ is —(CR^{2b}R^{2c})ₘ— the sum of m and n is not less than 2 and no more than 5;
or

### A-Y¹—X— is

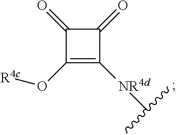

L is selected from

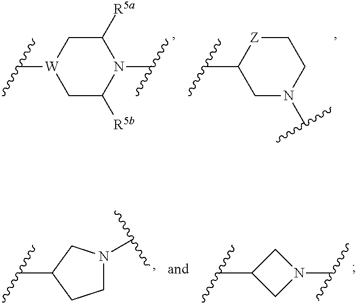

### W is CH or N;

Z is selected from CH₂, O and NR^{5c};
Y³ is selected from —O—(CR^{6a}R^{6b})—, —(CR^{6c}R^{6d})—O—, —CH═CH— and —CR^{6e}R^{6f}—CR^{6g}R^{6h}—;
R^{1a}, R^{1b}, R^{1c}, R^{1d} and R^{1e} are defined according to any one of
(a) R^{1b} and R^{1d} is halogen, and R^{1a}, R^{1c} and R^{1e} is H;
(b) R^{1c} is C₁₋₄haloalkyl, in particular CF₃, and R^{1a}, R^{1b}, R^{1d} and R^{1e} are H;
(c) R^{1b} is C₄alkyl, R^{1d} is halogen, and R^{1a}, R^{1c} and R^{1e} is H;
(d) R^{1b} is CN, R^{1d} is halogen, and R^{1a}, R^{1c} and R^{1e} is H; and
(e) R^{1a} and R^{1c} is halogen, and R^{1b}, R^{1d} and R^{1e} is H;
R² is selected from H, C₁₋₄alkyl and halogen;
R^{2a}, e, R^{2c}, R³, R^{4a}, R^{4b}, R^{4c}, R^{4d}, R^{5a}, R^{5b}, R^{5c}, R^{6a}, R^{6b}, R^{6c}, R^{6d}, R^{6e}, R^{6f}, R^{6g} and R^{6l} are independently selected from H and C₁₋₄alkyl.

### DEFINITIONS

“Halo” or “halogen”, as used herein, may be fluoro, chloro, bromo or iodo.

“C₁₋₄ alkyl”, as used herein, denotes straight chain or branched alkyl having 1-4 carbon atoms. If a different number of carbon atoms is specified, such as C₆ or C₃, then the definition is to be amended accordingly, such as “C₁-C₄ alkyl” will represent methyl, ethyl, propyl, isopropyl, butyl, isobutyl, sec-butyl and tert-butyl.

“C₁₋₄ haloalkyl”, as used herein, denotes straight chain or branched alkyl having 1-4 carbon atoms with at least one hydrogen substituted with a halogen. If a different number of carbon atoms is specified, such as C₆ or C₃, then the definition is to be amended accordingly, such as “C₁-C₄-Haloalkyl” will represent methyl, ethyl, propyl, isopropyl, butyl, isobutyl, sec-butyl and tert-butyl that have at least one hydrogen substituted with halogen, such as where the halogen is fluorine: CF₃CF₂—, (CF₃)₂CH—, CH₃—CF₂—, CF₃CF₂—, CF₃, CF₂H—, CF₃CF₂CHCF₃ or CF₃CF₂CF₂CF₂—.

“C₁₋₄ haloalkoxy” as used herein refers to an —O—C₁₋₄ alkyl group wherein C₁₋₄ alkyl is as defined herein and substituted with one or more halogen groups, e.g. —O—CF₃.

The term “a,” “an,” “the” and similar terms used in the context of the present invention (especially in the context of the claims) are to be construed to cover both the singular and plural unless otherwise indicated herein or clearly contradicted by the context.

As used herein, the term “subject” refers to an animal. Typically the animal is a mammal. A subject also refers to for example, primates (e.g., humans, male or female), cows, sheep, goats, horses, dogs, cats, rabbits, rats, mice, fish, birds and the like. In certain embodiments, the subject is a primate. In yet other embodiments, the subject is a human.

As used herein, the term “inhibit”, “inhibition” or “inhibiting” refers to the reduction or suppression of a given condition, symptom, or disorder, or disease, or a significant decrease in the baseline activity of a biological activity or process.

As used herein, the term “treat”, “treating” or “treatment” of any disease or disorder refers in one embodiment, to ameliorating the disease or disorder (i.e., slowing or arresting or reducing the development of the disease or at least one of the clinical symptoms thereof). In another embodiment “treat”, “treating” or “treatment” refers to alleviating or ameliorating at least one physical parameter including those which may not be discernible by the patient. In yet another embodiment, “treat”, “treating” or “treatment” refers to modulating the disease or disorder, either physically, (e.g., stabilization of a discernible symptom), physiologically, (e.g., stabilization of a physical parameter), or both. In yet another embodiment, “treat”, “treating” or “treatment” refers to preventing or delaying the onset or development or progression of the disease or disorder.

As used herein, a subject is “in need of” a treatment if such subject would benefit biologically, medically or in quality of life from such treatment.

As used herein, when one embodiment refers to several other embodiments by using the term “according to any one of”, for example “according to any one of embodiments 1 to 5”, then said embodiment refers not only to embodiments indicated by the integers such as 1 and 2 but also to embodiments indicated by numbers with a decimal component such as 1.1, 1.2 or 2.1, 2.2, 2.3. For example, “according to any one of embodiments 1 to 3” means according to any one of embodiments 1, 1.1, 2, 3, 3.1, 3.2, 3.3, 3.4, 3.5, 3.6, 3.7.

Various embodiments of the invention are described herein. It will be recognized that features specified in each embodiment may be combined with other specified features to provide further embodiments.

In embodiment 3 of the invention, there is provided a compound or salt according to any one of embodiments 1 to 2, wherein

R^{1a}, R^{1b}, R^{1c}, R^{1d} and R^{1e} are defined according to any one of
(a) R^{1b} is halogen, R^{1d} is halogen, CN, C₄alkyl, C₁₋₄haloalkyl or C₁₋₄haloalkoxy, and R^{1a}, R^{1c} and R^{1e} is H;
(b) R^{1b} is halogen, R^{1d} is halogen, CN, C₄alkyl, C₁₋₄haloalkyl or C₁₋₄haloalkoxy, R^{1c} is halogen, and R^{1a} and R^{1e} is H;
(c) R^{1b} is C₁₋₄alkyl, R^{1d} is C₁₋₄alkyl, C₄haloalkyl, C₁₋₄haloalkoxy or CN, R^{1a}, R^{1c} and R^{1e} is H;
(d) R^{1b} is CN, R^{1d} is C₁₋₄haloalkyl or C₁₋₄haloalkoxy, and R^{1a}, R^{1c} and R^{1e} is H;
(f) R^{1a} is halogen, R^{1c} is halogen, CN, C₄alkyl, C₁₋₄haloalkyl or C₁₋₄haloalkoxy, and R^{1b}, R^{1d} and R^{1e} is H; and
(g) R^{1c} is halogen, CN, C₄alkyl, C₁₋₄haloalkyl or C₁₋₄haloalkoxy, and R^{1a}, R^{1b} and R^{1e} are H, and R^{1d} is halogen, CN, C₄alkyl, C₁₋₄haloalkyl or C₁₋₄haloalkoxy, or H.

In embodiment 3.1 of the invention, there is provided a compound or salt according to any one of embodiments 1 to 2, wherein

R^{1a}, R^{1b}, R^{1c}, R^{1d} and R^{1e} are defined according to any one of
(a) R^{1b} is halogen, R^{1d} is halogen, CN, C₄alkyl, C₁₋₄haloalkyl or C₁₋₄haloalkoxy, and R^{1a}, R^{1c} and R^{1e} is H;
(c) R^{1b} is C₁₋₄alkyl, R^{1d} is C₁₋₄alkyl, C₄haloalkyl, C₁₋₄haloalkoxy or CN, R^{1a}, R^{1c} and R^{1e} is H;
(f) R^{1a} is halogen, R^{1c} is halogen, CN, C₄alkyl, C₁₋₄haloalkyl or C₁₋₄haloalkoxy, and R^{1b}, R^{1d} and R^{1e} is H; and
(g) R^{1c} is halogen, CN, C₄alkyl, C₁₋₄haloalkyl or C₁₋₄haloalkoxy, and R^{1a}, R^{1b} and R^{1e} are H, and R^{1d} is halogen, CN, C₄alkyl, C₁₋₄haloalkyl or C₁₋₄haloalkoxy, or H.

In embodiment 3.2 of the invention, there is provided a compound or salt according to any one of embodiments 1 to 2, wherein

R^{1a}, R^{1b}, R^{1c}, R^{1d} and R^{1e} are defined according to any one of
(a) R^{1b} is fluoro, chloro or bromo; R^{1d} is fluoro, chloro, bromo, CN, methyl, trifluoromethyl or trifluoromethoxy; and R^{1a}, R^{1c} and R^{1e} are H;
(c) R^{1b} is methyl; R^{1d} is methyl, trifluoromethyl, trifluoromethoxy or CN; R^{1a}, R^{1c} and R^{1e} are H;
(f) R^{1a} is fluoro, chloro or bromo; R^{1c} is fluoro, chloro, bromo, CN, methyl, trifluoromethyl or trifluoromethoxy; and R^{1b}, R^{1d} and R^{1e} are H; and
(g) R^{1c} is fluoro, chloro, bromo, CN, methyl, trifluoromethyl or trifluoromethoxy; and R^{1a}, R^{1b} and R^{1e} are H; and R^{1d} is fluoro, chloro, bromo, CN, methyl, trifluoromethyl, trifluoromethoxy, or H.

In embodiment 3.3 of the invention, there is provided a compound or salt according to any one of embodiments 1 to 2, wherein

R^{1b} is fluoro, chloro or bromo; R^{1d} is fluoro, chloro, bromo, CN, methyl, trifluoromethyl or trifluoromethoxy; and R^{1a}, R^{1c} and R^{1e} are H.

In embodiment 3.4 of the invention, there is provided a compound or salt according to any one of embodiments 1 to 2, wherein

R^{1b} is methyl; R^{1d} is methyl, trifluoromethyl, trifluoromethoxy or CN; R^{1a}, R^{1c} and R^{1e} are H.

In embodiment 3.5 of the invention, there is provided a compound or salt according to any one of embodiments 1 to 2, wherein

R^{1a} is fluoro, chloro or bromo; R^{1c} is fluoro, chloro, bromo, CN, methyl, trifluoromethyl or trifluoromethoxy; and R^{1b}, R^{1d} and R^{1e} are H.

In embodiment 3.6 of the invention, there is provided a compound or salt according to any one of embodiments 1 to 2, wherein

R^{1c} is fluoro, chloro, bromo, CN, methyl, trifluoromethyl or trifluoromethoxy; and R^{1a}, R^{1b} and R^{1e} are H; and R^{1d} is fluoro, chloro, bromo, CN, methyl, trifluoromethyl, trifluoromethoxy, or H.

In embodiment 3.7 of the invention, there is provided a compound or salt according to any one of embodiments 1 to 2, wherein R^{1b} and R^{1d} is halogen and R^{1a}, R^{1c} and R^{1e} is H.

In embodiment 4 of the invention, there is provided a compound or salt according to embodiment 3.7, wherein R^{1b} and R^{1d} is chloro and R^{1a}, R^{1c} and R^{1e} is H.

In embodiment 4.1 of the invention, there is provided a compound or salt according to any one of embodiments 1 to 3, wherein R^{1b} is CN, R^{1d} is methyl, and R^{1a}, R^{1c} and R^{1e} are H.

In embodiment 4.2 of the invention, there is provided a compound or salt according to any one of embodiments 1 to 3, wherein R^{1b} is fluoro, R^{1d} is chloro, and R^{1a}, R^{1c} and R^{1e} are H.

In embodiment 4.3 of the invention, there is provided a compound or salt according to any one of embodiments 1 to 3, wherein R^{1b} is chloro, R^{1c} is chloro, and R^{1a}, R^{1d} and R^{1e} are H.

In embodiment 4.4 of the invention, there is provided a compound or salt according to any one of embodiments 1 to 3, wherein R^{1b} is CN, R^{1d} is chloro, and R^{1a}, R^{1c} and R^{1e} are H.

In embodiment 4.5 of the invention, there is provided a compound or salt according to any one of embodiments 1 to 3, wherein R^{1b} is methyl, R^{1d} is methyl, and R^{1a}, R^{1c} and R^{1e} are H.

In embodiment 4.6 of the invention, there is provided a compound or salt according to any one of embodiments 1 to 3, wherein R^{1c} is CF³, and R^{1a}, R^{1b}, R^{1d} and R^{1e} are H.

In embodiment 4.7 of the invention, there is provided a compound or salt according to any one of embodiments 1 to 3, wherein R^{1b} is methyl, R^{1d} is chloro, and R^{1a}, R^{1c} and R^{1e} are H.

In embodiment 4.8 of the invention, there is provided a compound or salt according to any one of embodiments 1 to 3, wherein R^{1b} is methyl, R^{1d} is CF³, and R^{1a}, R^{1c} and R^{1e} are H.

In embodiment 4.9 of the invention, there is provided a compound or salt according to any one of embodiments 1 to 3, wherein R^{1b} is bromo, R^{1d} is CF³, and R^{1a}, R^{1c} and R^{1e} are H.

In embodiment 4.10 of the invention, there is provided a compound or salt according to any one of embodiments 1 to 3, wherein R^{1b} is CN, R^{1d} is CF³, and R^{1a}, R^{1c} and R^{1e} are H.

In embodiment 4.11 of the invention, there is provided a compound or salt according to any one of embodiments 1 to 3, wherein R^{1b} is OCF³, R^{1d} is chloro, and R^{1a}, R^{1c} and R^{1e} are H.

In embodiment 4.12 of the invention, there is provided a compound or salt according to any one of embodiments 1 to 3, wherein R^{1b} is chloro, R^{1c} is fluoro, R^{1d} is CN and R^{1a} and R^{1e} are H.

In embodiment 5 of the invention, there is provided a compound or salt according to any one of embodiments 1 to 4, wherein Y³ is selected from —O—(CH₂)—, (CH₂)—O—, —CH═CH—, —CH₂CH₂—, and —O—(CH₂—CH₂)—.

In embodiment 5.1 of the invention, there is provided a compound or salt according to any one of embodiments 1 to 4, wherein Y³ is —O—(CR^{6a}R^{6b})— or —(CR^{6c}R^{6d})—O—, particularly —O—(CR^{6a}R^{6b})—.

In embodiment 6 of the invention, there is provided a compound or salt according to any one of embodiments 1 to 5, wherein X is selected from —N(R³)—C(═O)— and —C(═O)—N(R³)—, in particular —N(H)—C(═O)— and —C(═O)—N(H)—

In embodiment 6.1 of the invention, there is provided a compound or salt according to any one of embodiments 1 to 5, wherein X is selected from —C(═O)—, —N(H)—C(═O)—, C(═O)—N(H)— and —C(═O)—N(CH₃)—.

In embodiment 7 of the invention, there is provided a compound or salt according to any one of embodiments 1 to 6, wherein L is selected from

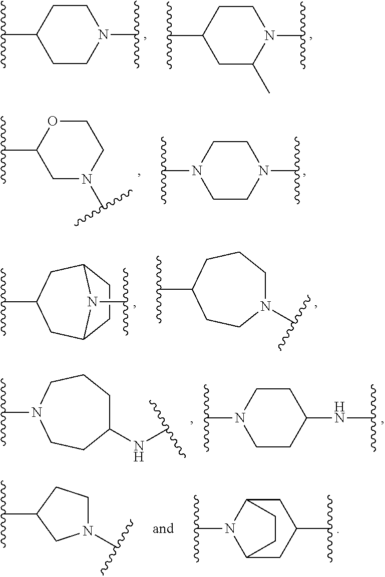

In embodiment 7.1 of the invention, there is provided a compound or salt according to any one of embodiments 1 to 6, wherein L is selected from

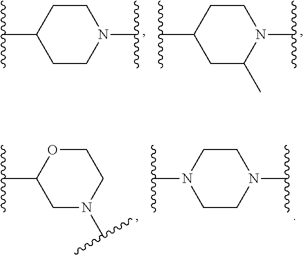

In embodiment 8 of the invention, there is provided a compound or salt according to any one of embodiments 1 to 7 with formula (II)

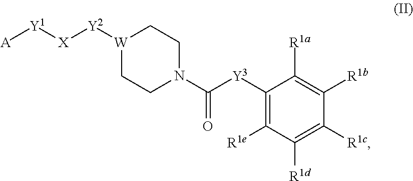

or a pharmaceutically acceptable salt thereof.

In embodiment 8.1 of the invention, there is provided a compound or salt according to any one of embodiments 1 to 7, wherein

Y¹ is —C(═O)—(CR^{2b}R^{2c})ₘ— or —C(OH)—(CR^{2b}R^{2c})ₘ—;
m is selected from 0, 1, 2, 3 and 4;
n is selected from 0, 1, 2 and 3; and wherein
the sum of m and n is not less than 2 and no more than 5.

In embodiment 9 of the invention, there is provided a compound or salt according to embodiment 8, wherein

m is selected from 2 and 3, and n is selected from 0 and 1; or
m is selected from 0 and 1, and n is selected from 2 and 3.

In embodiment 10 of the invention, there is provided a compound or salt according to embodiment 9, wherein

m is selected from 2 and 3, and n is 0.

In embodiment 11 of the invention, there is provided a compound or salt according to embodiment 10, wherein

m is 3, and n is 0.

In embodiment 12 of the invention, there is provided a compound or salt according to any one of embodiments 1 to 11, wherein

### X is —C(═O)—N(R³)—.

In embodiment 13 of the invention, there is provided a compound or salt according to any one of embodiments 1 to 12, wherein

A is selected from

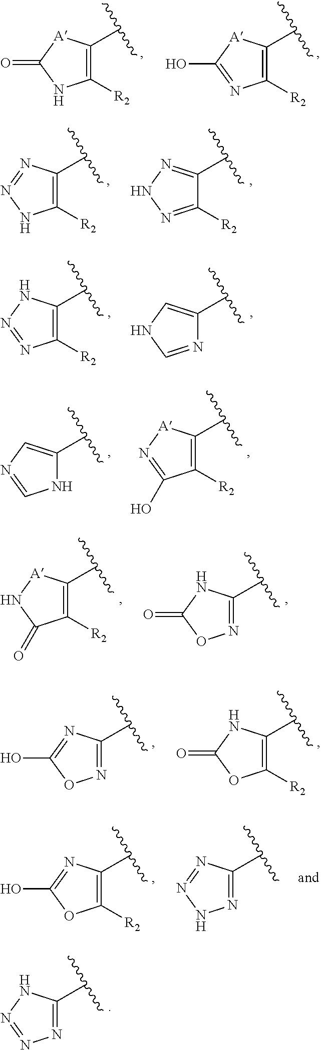

In embodiment 14 of the invention, there is provided a compound or salt according to embodiment 13, wherein

A is selected from

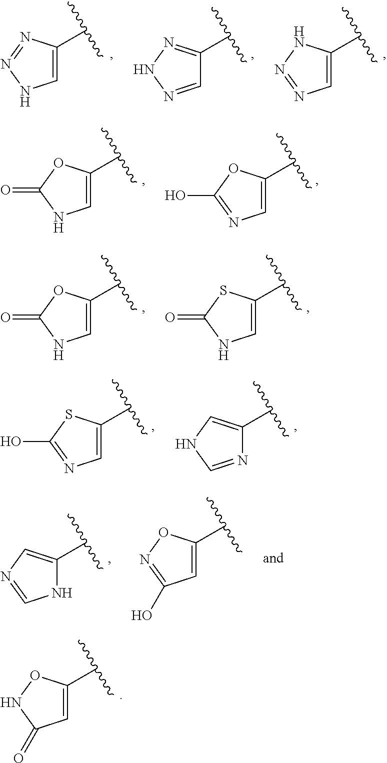

In embodiment 15 of the invention, there is provided a compound or salt according to embodiment 14, wherein A is

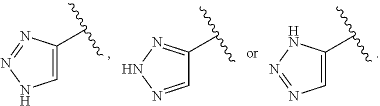 [SMILES: *c1c[nH]nn1 ; *c1cn[nH]n1 ; *c1cnn[nH]1]

In embodiment 16.2 of the invention, there is provided a compound or salt according to any one of embodiments 1 to 5, wherein A-Y¹—X—Y²-L- is selected from

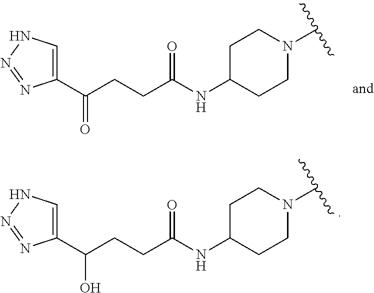 [SMILES: *N1CCC(NC(=O)CCC(=O)c2c[nH]nn2)CC1 ; *N1CCC(NC(=O)CCC(O)c2c[nH]nn2)CC1]

In embodiment 17 of the invention, there is provided a compound or salt according to any one of embodiments 1 to 6, wherein W is CH.

In embodiment 18 of the invention, there is provided a compound of formula (I)

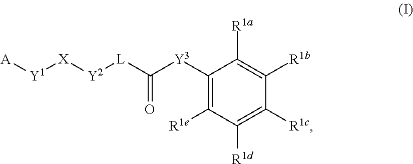

or a pharmaceutically acceptable salt thereof, wherein
A is selected from

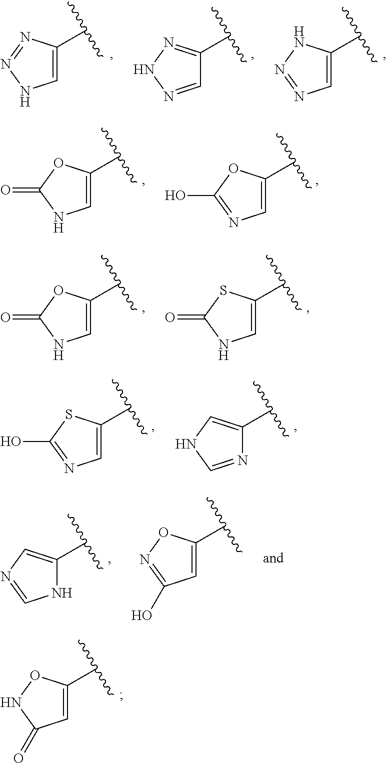

Y¹ is —C(═O)—(CR^{2b}R^{2c})ₙ— or —C(OH)—(CR^{2b}R^{2c})ₘ—;
X is selected from —C(═O)—, —N(R³)—C(═O)— and —C(═O)—N(R³)—;
Y² is —(CR^{4a}R^{4b})ₙ—;
m is selected from 0, 1, 2, 3 and 4;
n is selected from 0, 1, 2, 3, 4 and 5;
wherein when Y¹ is —(CR^{2b}R^{2c})ₙ— the sum of m and n is not less than 2 and no more than 5;
L is selected from

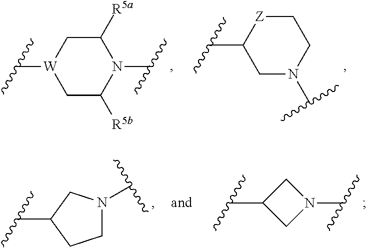

### W is CH or N;

Z is selected from CH₂, O and NR^{5c};
Y³ is selected from —O—(CR^{6a}R^{6b})—, (CR^{6c}R^{6d})—O—, —CH═CH—, CR^{6e}R^{6f}—CR^{6g}R^{6b}—, and —O—(CR⁶ⁱR^{6j}—CR^{6k}R^{6l})—;
R^{1a}, R^{1b}, R^{1c}, R^{1d} and R^{1e} are defined according to any one of
(a) R^{1b} is halogen; R^{1d} is halogen, CN, C₄alkyl, C₁₋₄haloalkyl or C₁₋₄haloalkoxy; and R^{1a}, R^{1c} and R^{1e} are H;
(b) R^{1b} is halogen; R^{1d} is halogen, CN, C₄alkyl, C₁₋₄haloalkyl or C₁₋₄haloalkoxy; R^{1c} is halogen; and R^{1a} and R^{1e} are H;
(c) R^{1b} is C₁₋₄alkyl; R^{1d} is C₁₋₄alkyl, C₄haloalkyl, C₁₋₄haloalkoxy or CN; R^{1a}, R^{1c} and R^{1e} are H;
(d) R^{1b} is CN; R^{1d} is C₁₋₄haloalkyl or C₁₋₄haloalkoxy; and R^{1a}, R^{1c} and R^{1e} are H;
(e) R^{1b} is C₁₋₄haloalkyl or C₁₋₄haloalkoxy; and R^{1a}, R^{1c} and R^{1e} are H; and R^{1d} is H or CN;
(f) R^{1a} is halogen; R^{1c} is halogen, CN, C₄alkyl, C₁₋₄haloalkyl or C₁₋₄haloalkoxy; and R^{1b}, R^{1d} and R^{1e} are H;
(g) R^{1c} is halogen, CN, C₄alkyl, C₁₋₄haloalkyl or C₁₋₄haloalkoxy; and R^{1a}, R^{1b} and R^{1e} are H; and R^{1d} is halogen, CN, C₄alkyl, C₄haloalkyl, C₁₋₄haloalkoxy, or H;
R^{2b}, R^{2c}, R³, R^{4a}, R^{4b}, R^{5a}, R^{5b}, R^{5c}, R^{6a}, R^{6b}, R^{6c}, R^{6d}, R^{6e}, R^{6f}, R^{6g}, R^{6h}, R⁶ⁱ, R^{6j}, R^{6k} and R^{6l} are independently selected from H and C₁₋₄alkyl.

In embodiment 19 of the invention, there is provided a compound of formula (II)

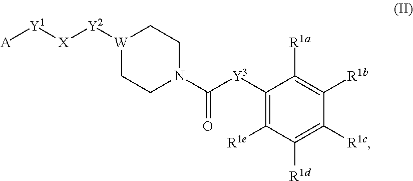

or a pharmaceutically acceptable salt thereof, wherein
A is selected from

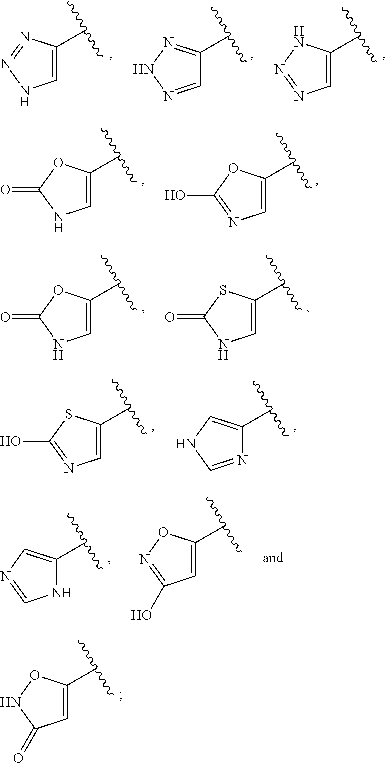

Y¹ is —C(═O)—(CR^{2b}R^{2c})ₘ— or —C(OH)—(CR^{2b}R^{2c})ₘ—;
X is selected from —N(R³)—C(═O)— and —C(═O)—N(R³)—;
Y² is —(CR^{4a}R^{4b})ₙ—;
m is selected from 0, 1, 2, 3 and 4;
n is selected from 0, 1, 2, 3, 4 and 5;
wherein the sum of m and n is not less than 2 and no more than 5;

### W is CH or N;

Y³ is selected from —O—(CR^{6a}R^{6b})— and —CH═CH—,
R^{1a}, R^{1b}, R^{1c}, R^{1d} and R^{1e} are defined according to any one of
(a) R^{1b} is halogen; R^{1d} is halogen, CN, C₄alkyl, C₁₋₄haloalkyl or C₁₋₄haloalkoxy; and R^{1a}, R^{1c} and R^{1e} are H;
(b) R^{1b} is CN; R^{1d} is C₁₋₄haloalkyl or C₁₋₄haloalkoxy; and R^{1a}, R^{1c} and R^{1e} are H;
R^{2b}, R^{2c}, R³, R^{4a}, R^{4b}, R^{4c}, R^{4d}, R^{6a} and R^{6b} are independently selected from H and C₁₋₄alkyl.

In embodiment 20 of the invention, there is provided a compound of formula (IV)

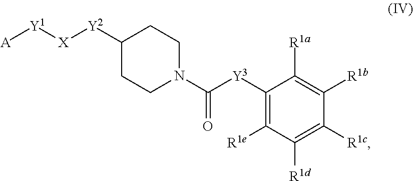

or a pharmaceutically acceptable salt thereof, wherein
A is selected from

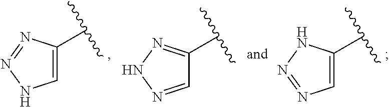 [SMILES: *c1c[nH]nn1 ; *c1cn[nH]n1 ; *c1cnn[nH]1]

Y¹ is —C(═O)—(CH₂)ₙ— or —C(OH)—(CH₂)ₘ—;
X is selected from —NH—C(═O)— and —C(═O)—NH—;
Y² is —(CH₂)ₙ—;
m is selected from 2 and 3, and n is selected from 0 and 1; or
m is selected from 0 and 1, and n is selected from 2 and 3;
Y³ is selected from —O—(CH₂)—,
R^{1a}, R^{1b}, R^{1c}, R^{1d} and R^{1e} are defined according to
(a) R^{1b} is chloro; R^{1d} is halogen and R^{1a}, R^{1c} and R^{1e} are H;
(b) R^{1b} is CN; R^{1d} is C₁₋₄haloalkyl or C₁₋₄haloalkoxy; and R^{1a}, R^{1c} and R^{1e} are H.

In embodiment 21 of the invention, there is provided a compound of formula (IV)

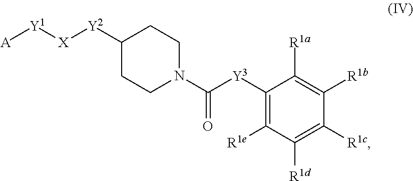

or a pharmaceutically acceptable salt thereof, wherein
A is selected from

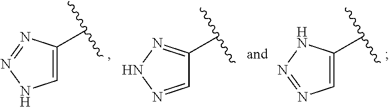 [SMILES: *c1c[nH]nn1 ; *c1cn[nH]n1 ; *c1cnn[nH]1]

Y¹ is —C(═O)—(CH₂)ₘ— or —C(OH)—(CH₂)ₘ—;

### X is —C(═O)—NH—;

Y² is —(CH₂)ₙ—;
m is selected from 2 and 3, and n is selected from 0 and 1;
Y³ is —O—(CH₂)—,
R^{1a}, R^{1b}, R^{1c}, R^{1d} and R^{1e} are defined according to
(a) R^{1b} and R^{1d} is chloro and R^{1a}, R^{1c} and R^{1e} are H; or
(b) R^{1b} is CN; R^{1d} is CF₃ or OCF₃; and R^{1a}, R^{1c} and R^{1e} are H.

In embodiment 22 of the invention, there is provided a compound of formula (IV)

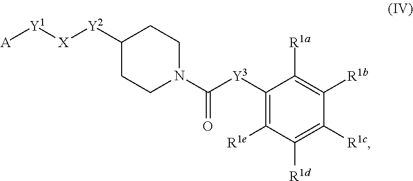

or a pharmaceutically acceptable salt thereof, wherein
A is selected from

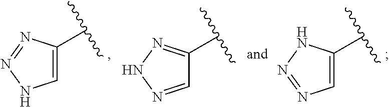 [SMILES: *c1c[nH]nn1 ; *c1cn[nH]n1 ; *c1cnn[nH]1]

Y¹ is —C(═O)—(CH₂)ₘ— or —C(OH)—(CH₂)ₘ—;

### X is —C(═O)—NH—;

Y² is —(CH₂)ₙ—;
m iS 3, and n is 0;
Y³ is —O—(CH₂)—,
R^{1a}, R^{1b}, R^{1c}, R^{1d} and R^{1e} are defined according to
(a) R^{1b} and R^{1d} is chloro and R^{1a}, R^{1c} and R^{1e} are H; or
(b) R^{1b} is CN; R^{1d} is CF₃; and R^{1a}, R^{1c} and R^{1e} are H.

In embodiment 23 of the invention, there is provided a compound or salt according to any one of embodiments 1 to 7, of formula (III)

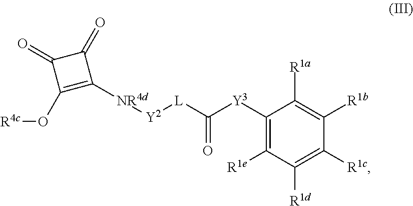

or a pharmaceutically acceptable salt thereof.

In embodiment 24 of the invention, there is provided a compound or salt according to embodiment 23, wherein

L is selected from

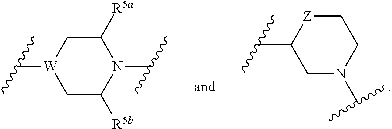

In embodiment 25 of the invention, there is provided a compound or salt according to embodiment 23 or 24, wherein

Y² is —(CR^{4a}R^{4b})ₙ— and n is 1 or 2, particularly 2.

In embodiment 26 of the invention, there is provided a compound or salt according to any one of embodiments 18 to 20, wherein

R^{4c} is methyl or ethyl and R^{4d} is methyl or H.

In embodiment 27 of the invention, there is provided a compound according to embodiment 1 selected from the group consisting of 3,5-dichlorobenzyl 4-(4-oxo-4-(1H-1,2,3-triazol-4-yl)butanamido)piperidine-1-carboxylate; 3,5-dichlorobenzyl 4-(4-hydroxy-4-(1H-1,2,3-triazol-4-yl)butanamido)piperidine-1-carboxylate;

or a pharmaceutically acceptable salt thereof.

In embodiment 28 of the invention, there is provided a compound according to embodiment 1 which is 3,5-dichlorobenzyl 4-(4-oxo-4-(1H-1,2,3-triazol-4-yl)butanamido)piperidine-1-carboxylate.

In embodiment 29 of the invention, there is provided a compound according to embodiment 1 which is 3,5-dichlorobenzyl 4-(4-hydroxy-4-(1H-1,2,3-triazol-4-yl)butanamido)piperidine-1-carboxylate.

The term “compounds of the (present) invention” or “a compound of the (present) invention” refers to a compound as defined in any one of embodiments 1 to 29.

The compounds of the present invention may be prepared by the routes described in the Examples or may be prepared according to known methods.

Within the scope of this text, only a readily removable group that is not a constituent of the particular desired end product of the compounds of the present invention is designated a “protecting group”, unless the context indicates otherwise. The protection of functional groups by such protecting groups, the protecting groups themselves, and their cleavage reactions are described for example in standard reference works, such as J. F. W. McOmie,

“Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, in “Methoden der organischen Chemie” (Methods of Organic Chemistry), Houben Weyl, 4th edition, Volume 15/I, Georg Thieme Verlag, Stuttgart 1974, in H.-D. Jakubke and H. Jeschkeit, “Aminosauren, Peptide, Proteine” (Amino acids, Peptides, Proteins), Verlag Chemie, Weinheim, Deerfield Beach, and Basel 1982, and in Jochen Lehmann, “Chemie der Kohlenhydrate: Monosaccharide und Derivate” (Chemistry of Carbohydrates: Monosaccharides and Derivatives), Georg Thieme Verlag, Stuttgart 1974. A characteristic of protecting groups is that they can be removed readily (i.e. without the occurrence of undesired secondary reactions) for example by solvolysis, reduction, photolysis or alternatively under physiological conditions (e.g. by enzymatic cleavage).

Salts of compounds of the present invention having at least one salt-forming group may be prepared in a manner known to those skilled in the art. For example, salts of compounds of the present invention having acid groups may be formed, for example, by treating the compounds with metal compounds, such as alkali metal salts of suitable organic carboxylic acids, e.g. the sodium salt of 2-ethylhexanoic acid, with organic alkali metal or alkaline earth metal compounds, such as the corresponding hydroxides, carbonates or hydrogen carbonates, such as sodium or potassium hydroxide, carbonate or hydrogen carbonate, with corresponding calcium compounds or with ammonia or a suitable organic amine, stoichiometric amounts or only a small excess of the salt-forming agent preferably being used. Acid addition salts of compounds of the present invention are obtained in customary manner, e.g. by treating the compounds with an acid or a suitable anion exchange reagent. Internal salts of compounds of the present invention containing acid and basic salt-forming groups, e.g. a free carboxy group and a free amino group, may be formed, e.g. by the neutralisation of salts, such as acid addition salts, to the isoelectric point, e.g. with weak bases, or by treatment with ion exchangers.

Salts can be converted into the free compounds in accordance with methods known to those skilled in the art. Metal and ammonium salts can be converted, for example, by treatment with suitable acids, and acid addition salts, for example, by treatment with a suitable basic agent.

Mixtures of isomers obtainable according to the invention can be separated in a manner known to those skilled in the art into the individual isomers; diastereoisomers can be separated, for example, by partitioning between polyphasic solvent mixtures, recrystallisation and/or chromatographic separation, for example over silica gel or by e.g. medium pressure liquid chromatography over a reversed phase column, and racemates can be separated, for example, by the formation of salts with optically pure salt-forming reagents and separation of the mixture of diastereoisomers so obtainable, for example by means of fractional crystallisation, or by chromatography over optically active column materials.

Intermediates and final products can be worked up and/or purified according to standard methods, e.g. using chromatographic methods, distribution methods, (re-) crystallization, and the like.

The following applies in general to all processes mentioned herein before and hereinafter. All the above-mentioned process steps can be carried out under reaction conditions that are known to those skilled in the art, including those mentioned specifically, in the absence or, customarily, in the presence of solvents or diluents, including, for example, solvents or diluents that are inert towards the reagents used and dissolve them, in the absence or presence of catalysts, condensation or neutralizing agents, for example ion exchangers, such as cation exchangers, e.g. in the H+ form, depending on the nature of the reaction and/or of the reactants at reduced, normal or elevated temperature, for example in a temperature range of from about −100° C. to about 190° C., including, for example, from approximately −80° C. to approximately 150° C., for example at from −80 to −60° C., at room temperature, at from −20 to 40° C. or at reflux temperature, under atmospheric pressure or in a closed vessel, where appropriate under pressure, and/or in an inert atmosphere, for example under an argon or nitrogen atmosphere.

At all stages of the reactions, mixtures of isomers that are formed can be separated into the individual isomers, for example diastereoisomers or enantiomers, or into any desired mixtures of isomers, for example racemates or mixtures of diastereoisomers, for example analogously to the methods described under “Additional process steps”.

The solvents from which those solvents that are suitable for any particular reaction may be selected include those mentioned specifically or, for example, water, esters, such as lower alkyl-lower alkanoates, for example ethyl acetate, ethers, such as aliphatic ethers, for example diethyl ether, or cyclic ethers, for example tetrahydrofuran or dioxane, liquid aromatic hydrocarbons, such as benzene or toluene, alcohols, such as methanol, ethanol or 1- or 2-propanol, nitriles, such as acetonitrile, halogenated hydrocarbons, such as methylene chloride or chloroform, acid amides, such as dimethylformamide or dimethyl acetamide, bases, such as heterocyclic nitrogen bases, for example pyridine or N-methylpyrrolidin-2-one, carboxylic acid anhydrides, such as lower alkanoic acid anhydrides, for example acetic anhydride, cyclic, linear or branched hydrocarbons, such as cyclohexane, hexane or isopentane, methycyclohexane, or mixtures of those solvents, for example aqueous solutions, unless otherwise indicated in the description of the processes. Such solvent mixtures may also be used in working up, for example by chromatography or partitioning.

The compounds of the present invention, including their salts, may also be obtained in the form of hydrates, or their crystals may, for example, include the solvent used for crystallization. Different crystalline forms may be present.

The invention relates also to those forms of the process in which a compound obtainable as an intermediate at any stage of the process is used as starting material and the remaining process steps are carried out, or in which a starting material is formed under the reaction conditions or is used in the form of a derivative, for example in a protected form or in the form of a salt, or a compound obtainable by the process according to the invention is produced under the process conditions and processed further in situ.

All starting materials, building blocks, reagents, acids, bases, dehydrating agents, solvents and catalysts utilized to synthesize the compounds of the present invention are either commercially available or can be produced by organic synthesis methods known to one of ordinary skill in the art.

As used herein, the term “an optical isomer” or “a stereoisomer” refers to any of the various stereo isomeric configurations which may exist for a given compound of the present invention and includes geometric isomers. It is understood that a substituent may be attached at a chiral center of a carbon atom. The term “chiral” refers to molecules which have the property of non-superimposability on their mirror image partner, while the term “achiral” refers to molecules which are superimposable on their mirror image partner. Therefore, the invention includes enantiomers, diastereomers or racemates of the compounds of the present invention. “Enantiomers” are a pair of stereoisomers that are non-superimposable mirror images of each other. A 1:1 mixture of a pair of enantiomers is a “racemic” mixture. The term is used to designate a racemic mixture where appropriate. “Diastereoisomers” are stereoisomers that have at least two asymmetric atoms, but which are not mirror-images of each other. The absolute stereochemistry is specified according to the Cahn-IngoId-Prelog R-S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Certain compounds of the present invention described herein may contain one or more asymmetric centers or axes and may thus give rise to enantiomers, diastereomers, and other stereoisomeric forms that may be defined, in terms of absolute stereochemistry, as (R)- or (S)-.

Depending on the choice of the starting materials and procedures, the compounds of the present invention may be present in the form of one of the possible isomers or as mixtures thereof, for example as pure optical isomers, or as isomer mixtures, such as racemates and diastereoisomer mixtures, depending on the number of asymmetric carbon atoms. The present invention is meant to include all such possible isomers, including racemic mixtures, diasteriomeric mixtures and optically pure forms. Optically active (R)- and (S)-isomers may be prepared using chiral synthons or chiral reagents, or resolved using conventional techniques. If the compound of the present invention contains a double bond, the substituent may be E or Z configuration. If the compound of the present invention contains a disubstituted cycloalkyl, the cycloalkyl substituent may have a cis- or trans-configuration. All tautomeric forms, for example for group A in embodiment 1, are also intended to be included.

As used herein, the terms “salt” or “salts” refers to an acid addition or base addition salt of a compound of the present invention. “Salts” include in particular “pharmaceutical acceptable salts”. The term “pharmaceutically acceptable salts” refers to salts that retain the biological effectiveness and properties of the compounds of the present invention and, which typically are not biologically or otherwise undesirable. In many cases, the compounds of the present invention are capable of forming acid and/or base salts by virtue of the presence of amino and/or carboxyl groups or groups similar thereto.

Pharmaceutically acceptable acid addition salts can be formed with inorganic acids and organic acids, e.g., acetate, aspartate, benzoate, besylate, bromide/hydrobromide, bicarbonate/carbonate, bisulfate/sulfate, camphorsulfonate, chloride/hydrochloride, chlortheophyllonate, citrate, ethandisulfonate, fumarate, gluceptate, gluconate, glucuronate, hippurate, hydroiodide/iodide, isethionate, lactate, lactobionate, laurylsulfate, malate, maleate, malonate, mandelate, mesylate, methylsulphate, naphthoate, napsylate, nicotinate, nitrate, octadecanoate, oleate, oxalate, palmitate, pamoate, phosphate/hydrogen phosphate/dihydrogen phosphate, polygalacturonate, propionate, stearate, succinate, sulfosalicylate, tartrate, tosylate and trifluoroacetate salts.

Thus, in embodiment 30, there is provided a pharmaceutically acceptable salt of a compound according to any one of embodiments 27 to 29, wherein the salt is selected from chloride/hydrochloride.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like. Pharmaceutically acceptable base addition salts can be formed with inorganic and organic bases.

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

The pharmaceutically acceptable salts of the compounds of the present invention can be synthesized from a basic or acidic moiety, by conventional chemical methods. Generally, such salts can be prepared by reacting free acid forms of the compounds of the present invention with a stoichiometric amount of the appropriate base (such as Na, Ca, Mg, or K hydroxide, carbonate, bicarbonate or the like), or by reacting free base forms of the compounds of the present invention with a stoichiometric amount of the appropriate acid. Such reactions are typically carried out in water or in an organic solvent, or in a mixture of the two. Generally, use of non-aqueous media like ether, ethyl acetate, ethanol, isopropanol, or acetonitrile is desirable, where practicable. Lists of additional suitable salts can be found, e.g., in “Remington's Pharmaceutical Sciences”, 20th ed., Mack Publishing Company, Easton, Pa., (1985); and in “Handbook of Pharmaceutical Salts: Properties, Selection, and Use” by Stahl and Wermuth (Wiley-VCH, Weinheim, Germany, 2002).

Any formula given herein is also intended to represent unlabeled forms as well as isotopically labeled forms of the compounds of the present invention. Isotopically labeled compounds of the present invention have structures depicted by the formulas given herein except that one or more atoms are replaced by an atom having a selected atomic mass or mass number. Examples of isotopes that can be incorporated into compounds of the present invention include isotopes of hydrogen, carbon, nitrogen, oxygen, phosphorous, fluorine, and chlorine, such as ²H, ³H, ¹¹C, ¹³C, ¹⁴C, ¹⁵N, ¹⁸F ³¹P, ³²P, ³⁵S, ³⁶Cl, ¹²⁵I respectively. The invention includes various isotopically labeled compounds of the present invention, for example those into which radioactive isotopes, such as ³H and ¹⁴C, or those into which non-radioactive isotopes, such as ²H and ¹³C are present. Such isotopically labelled compounds of the present invention are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound of the present invention may be particularly desirable for PET or SPECT studies. Isotopically-labeled compounds of the present invention can generally be prepared by conventional techniques known to those skilled in the art or by processes analogous to those described in the accompanying Generic Schemes, Examples and Preparations using an appropriate isotopically-labeled reagent in place of the non-labeled reagent previously employed.

Further, substitution with heavier isotopes, particularly deuterium (i.e., ²H or D) may afford certain therapeutic advantages resulting from greater metabolic stability, for example increased in vivo half-life or reduced dosage requirements or an improvement in therapeutic index. It is understood that deuterium in this context is regarded as a substituent of a compound of the present invention. The concentration of such a heavier isotope, specifically deuterium, may be defined by the isotopic enrichment factor. The term “isotopic enrichment factor” as used herein means the ratio between the isotopic abundance and the natural abundance of a specified isotope. If a substituent in a compound of the present invention is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

Pharmaceutically acceptable solvates in accordance with the invention include those wherein the solvent of crystallization may be isotopically substituted, e.g. D₂O, d₆-acetone, d₆-DMSO.

Compounds of the invention, i.e. compounds of the present invention that contain groups capable of acting as donors and/or acceptors for hydrogen bonds may be capable of forming co-crystals with suitable co-crystal formers. These co-crystals may be prepared from compounds of the present invention by known co-crystal forming procedures. Such procedures include grinding, heating, co-subliming, co-melting, or contacting in solution compounds of the present invention with the co-crystal former under crystallization conditions and isolating co-crystals thereby formed. Suitable co-crystal formers include those described in WO 2004/078163. Hence the invention further provides co-crystals comprising a compound of the present invention.

Any asymmetric atom (e.g., carbon or the like) of the compound(s) of the present invention can be present in racemic or enantiomerically enriched, for example the (R)-, (S)- or (R,S)-configuration. In certain embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess in the (R)- or (S)-configuration. Substituents at atoms with unsaturated double bonds may, if possible, be present in cis-(Z)- or trans-(E)-form.

Accordingly, as used herein a compound of the present invention can be in the form of one of the possible isomers, rotamers, atropisomers, tautomers or mixtures thereof, for example, as substantially pure geometric (cis or trans) isomers, diastereomers, optical isomers (antipodes), racemates or mixtures thereof.

Any resulting mixtures of isomers can be separated on the basis of the physicochemical differences of the constituents, into the pure or substantially pure geometric or optical isomers, diastereomers, racemates, for example, by chromatography and/or fractional crystallization.

Any resulting racemates of final products or intermediates can be resolved into the optical antipodes by known methods, e.g., by separation of the diastereomeric salts thereof, obtained with an optically active acid or base, and liberating the optically active acidic or basic compound. In particular, a basic moiety may thus be employed to resolve the compounds of the present invention into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-10-sulfonic acid. Racemic products can also be resolved by chiral chromatography, e.g., high pressure liquid chromatography (HPLC) using a chiral adsorbent.

Furthermore, the compounds of the present invention, including their salts, can also be obtained in the form of their hydrates, or include other solvents used for their crystallization. The compounds of the present invention may inherently or by design form solvates with pharmaceutically acceptable solvents (including water); therefore, it is intended that the invention embrace both solvated and unsolvated forms. The term “solvate” refers to a molecular complex of a compound of the present invention (including pharmaceutically acceptable salts thereof) with one or more solvent molecules. Such solvent molecules are those commonly used in the pharmaceutical art, which are known to be innocuous to the recipient, e.g., water, ethanol, and the like. The term “hydrate” refers to the complex where the solvent molecule is water.

The compounds of the present invention, including salts, hydrates and solvates thereof, may inherently or by design form polymorphs.

The compounds of the present invention in free form or in salt form, exhibit valuable pharmacological properties, e.g. as indicated in in vitro tests as provided herein, and are therefore indicated for therapy or for use as research chemicals, e.g. as tool compounds.

Thus, in embodiment 31, there is provided a compound according to any one of embodiments 1 to 29 for use in medicine.

The compounds according to any one of embodiments 1 to 29 are potent inhibitors of ATX (see IC₅₀ data disclosed herein). The compounds of the present invention are hence useful in the treatment of an ATX-dependent or ATX-mediated disease or condition. The compounds according to any one of embodiments 1 to 29 have favourable pharmacokinetic properties, particularly following oral administration, more particularly at higher doses. The compounds according to any one of embodiments 1 to 29 have particularly favourable solubility and absorption profiles.

Thus, in embodiment 32, there is provided a compound according to any one of embodiments 1 to 29 for use in the treatment of an ATX-dependent or ATX-mediated disease or condition. In embodiment 33, there is provided the use of a compound according to any one of embodiments 1 to 34 in the treatment of an ATX-dependent or ATX-mediated disease or condition. In embodiment 34, there is provided the use of a compound according to any one of embodiments 1 to 29 in the manufacture of a medicament for the treatment of an ATX-dependent or ATX-mediated disease or condition. In embodiment 35, there is provided a method of treating an ATX-dependent or ATX-mediated disease or condition comprising administering to the subject a therapeutically effective amount of a compound according to any one of embodiments 1 to 29.

Hence, in a further embodiment 36, the compounds of the invention are useful for the treatment of a disease or condition according to embodiments 32, 33, 34 and 35, wherein the disease or condition is selected from fibrosis, pruritus, cirrhosis, cancer, diabetes, kidney diseases, asthma, COPD and pain.

In embodiment 37, the compounds of the invention are useful for the treatment of a disease or condition according to embodiment 36, wherein the disease or condition is selected from pulmonary fibrosis, idiopathic pulmonary fibrosis, a diffuse parenchymal interstitial lung disease including iatrogenic drug-induced fibrosis, occupational and/or environmental induced fibrosis (Farmer lung), radiation induced fibrosis, bleomycin induced pulmonary fibrosis, asbestos induced pulmonary fibrosis, acute respiratory distress syndrome (ARDS), kidney fibrosis, tubulointerstitium fibrosis, gut fibrosis, liver fibrosis, alcohol induced liver fibrosis, toxic/drug induced liver fibrosis, infection induced liver fibrosis, viral induced liver fibrosis, cutaneous fibrosis, spinal cord injury/fibrosis, myelofibrosis, renal fibrosis, skin fibrosis, ocular fibrosis, post-transplant fibrosis, hepatic fibrosis with or without cirrhosis, cardiac fibrosis, neuropathic pruritus, neurogenic pruritus, psychogenic pruritus, cholestatic pruritus, primary biliary cirrhosis, liver cirrhosis, breast cancer, pancreatic cancer, ovarian cancer, prostate cancer, glioblastoma, bone cancer, colon cancer, bowel cancer, head and neck cancer, diabetes, polycystic kidney disease, acute kidney injury, chronic kidney disease, asthma, COPD, neuropathic pain and cancer pain.

In embodiment 38, the compounds of the invention are useful for the treatment of a disease or condition according to embodiment 37, wherein the disease or condition is selected from idiopathic pulmonary fibrosis, breast cancer, pancreatic cancer, prostate cancer, cholestatic pruritus, primary biliary cirrhosis and polycystic kidney disease, particularly idiopathic pulmonary fibrosis.

The compounds of the invention will be typically formulated as pharmaceutical compositions.

Thus, in embodiment 39 of the invention, the present invention provides a pharmaceutical composition comprising a compound according to any one of embodiments 1 to 29, or a pharmaceutically acceptable salt thereof, and a pharmaceutically acceptable carrier.

The pharmaceutical composition can be formulated for particular routes of administration such as oral administration, parenteral administration, and rectal administration, etc. In addition, the pharmaceutical compositions of the present invention can be made up in a solid form (including without limitation capsules, tablets, pills, granules, powders or suppositories), or in a liquid form (including without limitation solutions, suspensions or emulsions). The pharmaceutical compositions can be subjected to conventional pharmaceutical operations such as sterilization and/or can contain conventional inert diluents, lubricating agents, or buffering agents, as well as adjuvants, such as preservatives, stabilizers, wetting agents, emulsifers and buffers, etc.

Typically, the pharmaceutical compositions are tablets or gelatin capsules comprising the active ingredient together with

a) diluents, e.g., lactose, dextrose, sucrose, mannitol, sorbitol, cellulose and/or glycine;
b) lubricants, e.g., silica, talcum, stearic acid, its magnesium or calcium salt and/or polyethyleneglycol; for tablets also
c) binders, e.g., magnesium aluminum silicate, starch paste, gelatin, tragacanth, methylcellulose, sodium carboxymethylcellulose and/or polyvinylpyrrolidone; if desired
d) disintegrants, e.g., starches, agar, alginic acid or its sodium salt, or effervescent mixtures; and/or
e) absorbents, colorants, flavors and sweeteners.

Tablets may be either film coated or enteric coated according to methods known in the art.

Suitable compositions for oral administration include an effective amount of a compound of the present invention in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets may contain the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Certain injectable compositions are aqueous isotonic solutions or suspensions, and suppositories are advantageously prepared from fatty emulsions or suspensions. Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1-75%, or contain about 1-50%, of the active ingredient.

Suitable compositions for transdermal application include an effective amount of a compound of the invention with a suitable carrier. Carriers suitable for transdermal delivery include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound of the skin of the host at a controlled and predetermined rate over a prolonged period of time, and means to secure the device to the skin.

Suitable compositions for topical application, e.g., to the skin and eyes, include aqueous solutions, suspensions, ointments, creams, gels or sprayable formulations, e.g., for delivery by aerosol or the like. Such topical delivery systems will in particular be appropriate for dermal application, e.g., for the treatment of skin cancer, e.g., for prophylactic use in sun creams, lotions, sprays and the like. They are thus particularly suited for use in topical, including cosmetic, formulations well-known in the art. Such may contain solubilizers, stabilizers, tonicity enhancing agents, buffers and preservatives.

As used herein a topical application may also pertain to an inhalation or to an intranasal application. They may be conveniently delivered in the form of a dry powder (either alone, as a mixture, for example a dry blend with lactose, or a mixed component particle, for example with phospholipids) from a dry powder inhaler or an aerosol spray presentation from a pressurised container, pump, spray, atomizer or nebuliser, with or without the use of a suitable propellant.

Where the inhalable form of the active ingredient is an aerosol composition, the inhalation device may be an aerosol vial provided with a valve adapted to deliver a metered dose, such as 10 to 100 μl, e.g. 25 to 50 μl, of the composition, i.e. a device known as a metered dose inhaler. Suitable such aerosol vials and procedures for containing within them aerosol compositions under pressure are well known to those skilled in the art of inhalation therapy. For example, an aerosol composition may be administered from a coated can, for example as described in EP-A-0642992. Where the inhalable form of the active ingredient is a nebulizable aqueous, organic or aqueous/organic dispersion, the inhalation device may be a known nebulizer, for example a conventional pneumatic nebulizer such as an airjet nebulizer, or an ultrasonic nebulizer, which may contain, for example, from 1 to 50 ml, commonly 1 to 10 ml, of the dispersion; or a hand-held nebulizer, sometimes referred to as a soft mist or soft spray inhaler, for example an electronically controlled device such as an AERx (Aradigm, US) or Aerodose (Aerogen), or a mechanical device such as a RESPIMAT (Boehringer Ingelheim) nebulizer which allows much smaller nebulized volumes, e.g. 10 to 100 μl, than conventional nebulizers. Where the inhalable form of the active ingredient is the finely divided particulate form, the inhalation device may be, for example, a dry powder inhalation device adapted to deliver dry powder from a capsule or blister containing a dry powder comprising a dosage unit of (A) and/or (B) or a multidose dry powder inhalation (MDPI) device adapted to deliver, for example, 3-25 mg of dry powder comprising a dosage unit of (A) and/or (B) per actuation. The dry powder composition preferably contains a diluent or carrier, such as lactose, and a compound that helps to protect against product performance deterioration due to moisture e.g. magnesium stearate. Suitable such dry powder inhalation devices include devices disclosed in U.S. Pat. No. 3,991,761 (including the AEROLIZER™ device), WO 05/113042 (including the BREEZHALER™ device), WO 97/20589 (including the CERTIHALER™ device), WO 97/30743 (including the TWISTHALER™ device), WO 05/37353 (including the GYROHALER™ device), U.S. Pat. No. 6,536,427 (including the DISKUS™ device), WO 97/25086 (including the DISKHALER™ device), WO 95/14089 (including the GEMINI™ device), WO 03/77979 (including the PROHALER™ device), and also the devices disclosed in WO 08/51621, WO 09/117112 and US 2005/0183724.

Hence, the invention also includes (A) a compound of the present invention, or a pharmaceutically acceptable salt thereof, in inhalable form; (B) an inhalable medicament comprising a compound of the present invention in inhalable form together with a pharmaceutically acceptable carrier in inhalable form; (C) a pharmaceutical product comprising a compound of the present invention in inhalable form in association with an inhalation device; and (D) an inhalation device containing a compound of the present invention in inhalable form.

Dosages of agents of the invention employed in practising the present invention will of course vary depending, for example, on the particular condition to be treated, the effect desired and the mode of administration. In general, suitable daily dosages for administration by inhalation are of the order of 0.0001 to 30 mg/kg, typically 0.01 to 10 mg per patient, while for oral administration suitable daily doses are of the order of 0.01 to 100 mg/kg.

The present invention further provides anhydrous pharmaceutical compositions and dosage forms comprising the compounds of the present invention as active ingredients, since water may facilitate the degradation of certain compounds.

Anhydrous pharmaceutical compositions and dosage forms of the invention can be prepared using anhydrous or low moisture containing ingredients and low moisture or low humidity conditions. An anhydrous pharmaceutical composition may be prepared and stored such that its anhydrous nature is maintained. Accordingly, anhydrous compositions are packaged using materials known to prevent exposure to water such that they can be included in suitable formulary kits. Examples of suitable packaging include, but are not limited to, hermetically sealed foils, plastics, unit dose containers (e. g., vials), blister packs, and strip packs.

The invention further provides pharmaceutical compositions and dosage forms that comprise one or more agents that reduce the rate by which the compound of the present invention as an active ingredient will decompose. Such agents, which are referred to herein as “stabilizers,” include, but are not limited to, antioxidants such as ascorbic acid, pH buffers, or salt buffers, etc.

The compound of the present invention may be administered either simultaneously with, or before or after, one or more other therapeutic agent. The compound of the present invention may be administered separately, by the same or different route of administration, or together in the same pharmaceutical composition as the other agents.

In one embodiment, the invention provides a product comprising a compound of the present invention and at least one other therapeutic agent as a combined preparation for simultaneous, separate or sequential use in therapy. In one embodiment, the therapy is the treatment of a disease or condition mediated by blockade of the epithelial sodium channel. Products provided as a combined preparation include a composition comprising the compound of the present invention and the other therapeutic agent(s) together in the same pharmaceutical composition, or the compound of the present invention and the other therapeutic agent(s) in separate form, e.g. in the form of a kit.

Thus, in embodiment 40, the invention provides a pharmaceutical composition comprising a compound according to any one of embodiments 1 to 29 and one or more therapeutically active co-agent. Optionally, the pharmaceutical composition may comprise a pharmaceutically acceptable excipient, as described above.

In one embodiment, the invention provides a kit comprising two or more separate pharmaceutical compositions, at least one of which contains a compound of the present invention. In one embodiment, the kit comprises means for separately retaining said compositions, such as a container, divided bottle, or divided foil packet. An example of such a kit is a blister pack, as typically used for the packaging of tablets, capsules and the like.

The kit of the invention may be used for administering different dosage forms, for example, oral and parenteral, for administering the separate compositions at different dosage intervals, or for titrating the separate compositions against one another. To assist compliance, the kit of the invention typically comprises directions for administration.

In embodiment 41 of the invention, there is provided a pharmaceutical combination, comprising:

a therapeutically effective amount of the compound according to any one of embodiments 1 to 29, or a pharmaceutically acceptable salt thereof, and one or more therapeutically active co-agent.

In embodiment 43 of the invention, there is provided a pharmaceutical combination according to embodiment 41, wherein the therapeutically active co-agent is selected from immunosuppresants, analgesics, anti-cancer agent, anti-inflammatories, chemokine receptor antagonists, bronchodilators, leukotriene receptor antagonists, leukotriene formation inhibitors, monoacylglycerol kinase inhibitors, phospholipase A1 inhibitors, phospholipase A2 inhibitors, lysophospholipase D (lysoPLD) inhibitors, decongestants, antihistamines, mucolytics, anticholinergics, antitussives, expectorants, and 13-2 agonists.

Suitable anti-inflammatory drugs include steroids, for example corticosteroids. Suitable steroids include budesonide, beclamethasone (e.g. dipropionate), butixocort (e.g. propionate), ciclesonide, ciclesonide, dexamethasone, flunisolide, fluticasone (e.g. propionate or furoate), methyl prednisolone, mometasone (e.g. furoate), prednisolone, rofleponide, and triamcinolone (e.g. acetonide). In certain preferred embodiments the steroid is long-acting corticosteroids such as budesonide, ciclesonide, fluticasone propionate, fluticasone furoate or mometasone furoate.

Suitable β₂-agonists include arformoterol (e.g. tartrate), abediterol, albuterol/salbutamol (e.g. racemate or single enantiomer such as the R-enantiomer, or salt thereof especially sulfate), bambuterol, bitolterol (e.g. mesylate), carmoterol, clenbuterol, etanterol, fenoterol (e.g. racemate or single enantiomer such as the R-enantiomer, or salt thereof especially hydrobromide), flerbuterol, arformoterol (e.g. tartrate), formoterol (e.g. racemate or single diastereomer such as the R,R-diastereomer, or salt thereof especially fumarate or fumarate dihydrate), indacaterol (e.g. racemate or single enantiomer such as the R-enantiomer, or salt thereof especially maleate, acetate or xinafoate), metaproterenol, milveterol (e.g. hydrochloride), naminterol, olodaterol (e.g. racemate or single enantiomer such as the R-enantiomer, or salt thereof especially hydrochloride), pirbuterol (e.g. acetate), procaterol, reproterol, salmefamol, salmeterol (e.g. racemate or single enantiomer such as the R-enantiomer, or salt thereof especially xinafoate), terbutaline (e.g. sulphate) and vilanterol (or a salt thereof especially trifenatate. In certain preferred embodiments the β₂-agonist is an ultra-long-acting β₂-agonist such as indacaterol, or potentially carmoterol, milveterol, olodaterol, or vilanterol. A preferred embodiment one of the second active ingredients is indacaterol (i.e. (R)-5-[2-(5,6-diethyl-indan-2-ylamino)-1-hydroxyethyl]-8-hydroxy-1H-quinolin-2-one) or a salt thereof. This is a β₂-adrenoceptor agonist that has an especially long duration of action (i.e. over 24 hours) and a short onset of action (i.e. about 10 minutes). This compound is prepared by the processes described in international patent applications WO 2000/75114 and WO 2005/123684. It is capable of forming acid addition salts, particularly pharmaceutically acceptable acid addition salts. A preferred salt of (R)-5-[2-(5,6-diethyl-indan-2-ylamino)-1-hydroxyethyl]-8-hydroxy-1H-quinolin-2-one is the maleate salt. Another preferred salt is (R)-5-[2-(5,6-diethyl-indan-2-ylamino)-1-hydroxyethyl]-8-hydroxy-1H-quinolin-2-one acetate. Another preferred salt is (R)-5-[2-(5,6-diethyl-indan-2-ylamino)-1-hydroxyethyl]-8-hydroxy-1H-quinolin-2-one xinafoate.

Suitable bronchodilatory drugs include anticholinergic or antimuscarinic agents, such as aclidinium (e.g. bromide), BEA-2108 (e.g. bromide), BEA-2180 (e.g. bromide), CHF-5407, darifenacin (e.g. bromide), darotropium (e.g. bromide), glycopyrrolate (e.g. racemate or single enantiomer, or salt thereof especially bromide), dexpirronium (e.g. bromide), ipratropium (e.g. bromide), otilonium (e.g. bromide), oxitropium (e.g. bromide), oxybutynin, pirenzepine, revatropate (e.g. hydrobromide), solifenacin (e.g. succinate), terodiline, umeclidinium (e.g. bromide), AZD-8683, tiotropium (e.g. bromide), tolterodine (e.g. tartrate), trospium (e.g. chloride), and those described in WO06/048225, WO06/066928 and WO06/066929. In certain preferred embodiments the muscarinic antagonists is long-acting muscarinic antagonist such as darotropium bromide, glycopyrrolate or tiotropium bromide.

Suitable dual anti-inflammatory and bronchodilatory drugs include dual beta-2 adrenoceptor agonist/muscarinic antagonists such as GSK-961081 (e.g. succinate) and AZD-2115.

Suitable antihistamine drug substances include cetirizine hydrochloride, acetaminophen, clemastine fumarate, promethazine, loratidine, desloratidine, diphenhydramine and fexofenadine hydrochloride, activastine, astemizole, azelastine, ebastine, epinastine, mizolastine and tefenadine, as well as those disclosed in JP 2004107299, WO 03/099807 and WO 04/026841.

### EXAMPLES

### Biocatalytic synthesis of 3,5-Dichlorobenzyl 4-(4-(1H-1,2,3-triazol-4-yl)butanamido)piperidine-1-carboxylate (Example 17) metabolites

Materials used:

Modified Terrific Broth 2 (MTB-2):

| Component | Concentration | | Supplier |
|---|---|---|---|
| Bacto Yeast Extract | 24 | g/L | Difco 212730 |
| Casein Digest | 14 | g/L | Difco 211610 |
| K2HPO4 | 7.36 | g/L | |
| KH2PO4 | 6.45 | g/L | |
| Glycerol | 10 | g/L | |

All components were dissolved in distilled water. pH was adjusted to 6.8. 1 mL/L thiamin-trace element solution was added (see below).

Trace element solution:

| | | |
|---|---|---|
| Fe (III) citrate | 24.5 | g/L |
| HCl conc. | 100 | mL/L |
| ZnCl2 | 1.31 | g/L |
| CoCl2 × 6 H2O | 2.00 | g/L |
| Na2MoO4 × 2 H2O | 2.00 | g/L |
| CaCl2 × 2 H2O | 1.00 | g/L |
| CuCl2 × 2 H20 | 1.27 | g/L |
| H3BO3 | 0.5 | g/L |

All components were dissolved in distilled water.

Thiamin-trace element solution:

3.37 g Thiamin (Vitamin B1) were dissolved in 7.5 mL distilled water. 2.5 mL trace element solution was added.

| | | |
|---|---|---|
| Component | Concentration | Supplier |

LB medium:

| | | |
|---|---|---|
| LB Broth | 25 g/L | Difco 244620 |

Antibiotics:

| | | |
|---|---|---|
| Ampicillin sodium | 50 mg/L | Sigma A9518 |
| Chloramphenicol | 25 mg/L | Calbiochem 220551 |

Absorber resins:

| | |
|---|---|
| Amberlite XAD-16N | Dow Chemical Company |
| Isolute HM-N bulk | Biotage 9800-5000 |

### Example 1

### 3,5-dichlorobenzyl 4-(4-oxo-4-(1H-1,2,3-triazol-4-yl)butanamido)piperidine-1-carboxylate

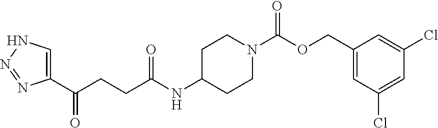 [SMILES: O=C(CCC(=O)c1c[nH]nn1)NC1CCN(C(=O)OCc2cc(Cl)cc(Cl)c2)CC1]

and

### Example 2

### 3,5-dichlorobenzyl 4-(4-hydroxy-4-(1H-1,2,3-triazol-4-yl)butanamido)piperidine-1-carboxylate

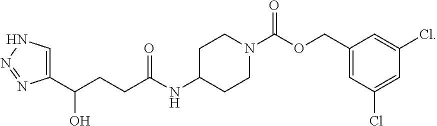 [SMILES: O=C(CCC(O)c1c[nH]nn1)NC1CCN(C(=O)OCc2cc(Cl)cc(Cl)c2)CC1]

### Introduction

The biocatalytic synthesis of Example 1 and 2 was carried out applying recombinant human CYP3A4 expressed in E. coli JM109 together with human NADPH-P450 reductase (CPR) and Cytochrome b5. Cells are stored as glycerol cultures at −80° C. Before application as whole cell biocatalysts the cells were cultivated in a wave bag bioreactor as described below.

### Fermentation:

Pre-culture: 200 ml LB medium (lysogeny broth) were filled into 1 litre Erlenmeyer flasks, supplemented with 50 mg/L ampicillin and 25 mg/L chloramphenicol, and inoculated with E. coli JM109 containing the recombinant genes for CYP3A4, CPR and Cytochrome b5. 3 Flasks were incubated over night at 37° C. and 160 rpm.

Main culture: A 50 litre wave bag bioreactor was filled with 25 litre sterilized MTB-2 medium and supplemented with 50 mg/L ampicillin and 25 mg/L chloramphenicol for plasmid selection. 600 mL of the pre-culture were transferred into the wave bag bioreactor. The fermentation conditions are shown in the following table:

| Temp | rocks | tilt | Air flow rate | Oxygen |
|---|---|---|---|---|
| [° C.] | [min−1] | angle | [mL/min] | content air |
| 30 | 39 | 9.8° | 450 | 10% |

After 4.5 hours an optical density of 0.98 at 600 nm was reached and the expression of recombinant genes was induced by adding 1 mM isopropyl β-D-1-thiogalactopyranoside and 0.5 mM 5-aminolevulinic acid hydrochloride. The temperature was reduced to 28° C. and the fermentation was continued for 19 hours (over night).

Harvesting: The cells were harvested by centrifugation (5 min at 4° C. and 12200×g). The supernatant was discarded and the pellets were resuspended in ice-cold PSE buffer. After a second centrifugation step the pellets were again suspended in ice-cold PSE buffer. 125 g XAD-16 were added to the cell suspension and stirred by a paddle agitator in order to remove by-products such as indole. Indole removal was monitored by HPLC-UV. After 40 min XAD-16 was removed by filtration. Additional PSE buffer was added to the cell suspension to achieve a final optical density of OD600 nm=100. The cell suspension was stored at −80° C. until usage for preparative biotransformation.

PSE buffer: 6.8 g/L KH2PO4, 85.6 g/L Sucrose, 0.9 g/L EDTA-Na in water, adjust pH to 7.5 with NaOH.

### Preparative Biotransformation:

340 mL of recombinant E. coli JM109 were thawed and filled in a 10 L wave fermenter. 1.36 L PSE buffer were added to achieve a final OD of 20. Furthermore, 83 mL of sodium citrate 46% (w/v) were added. The reaction was started by adding 50 mL NVP-LNC731 stock solution (10 mg/mL in DMSO) to achieve a final concentration of 0.3 mg/mL. The biotransformation conditions are shown in the following table:

| Temp | rocks | tilt | Air flow rate | Oxygen |
|---|---|---|---|---|
| [° C.] | [min−1] | angle | [L/min] | content air |
| 30 | 42 | 10.4° | 3 | 10% |

The biotransformation process was monitored by HPLC-UV. After 4 hours the reaction was stopped by pumping the reaction mixture from the wave bioreactor bag into a 5 L bottle. The bottle was stored at 4° C. until downstream processing.

### Downstream Processing:

90 g XAD-16 were added to the biotransformation mixture and stirred for 1 hour in order to absorb the biotransformation products. The extraction process was monitored by HPLC-UV. When the extraction with XAD-16 was completed, the XAD-16 material was filtered with gaze and washed with distilled water. The XAD-16 material was filled into a glass column. The column was washed several times with 2-propanol and acetonitrile/methanol 50/50 (% v/v) in order to elute the biotransformation products. The elution process was monitored by HPLC-UV analysis. The elution fractions were combined and the solvent was evaporated in a rotavapor at 40° C. and pressure between 70 and 130 mbar until the volume was reduced to 50 mL.

30 mL isolute HM-N were added and the mixture was frozen in dry-ice acetone until a fine solid layer was uniformly deposited in the walls of the flask. The mixture was lyophilized for 24 hours at a pressure below 0.1 mbar until a dry powder was obtained.

The isolute material comprising the biotransformation products was filled into a cartridge connected with a Armen SPOT Liquid Chromatography Flash device. The products were pre-purified by RP C18 Flash chromatography applying a water/acetonitrile gradient. The resulting fractions were analyzed by HPLC-UV and LC-MS. Fractions containing the biotransformation products were combined and further purified by supercritical fluid chromatography.

After purification, 9.7 mg of Example 1 and 38.8 mg of Example 2 were produced. The structures of Example 1 and 2 were elucidated by NMR and LC/MS (see below).

### NMR Experimental Conditions:

The NMR sample was prepared by dissolving Example 1 and 2 in ca 40 μl DMSO. The NMR spectra (¹H, ¹³C, ²D) were measured at 26° C. on a Bruker AVANCE spectrometer (600 MHz proton frequency) equipped with a 1.7 mm ¹H{¹³C, ¹⁵N} CryoProbe™. ¹H and ¹³C shifts were referenced internally to the solvent signals at 2.50 ppm and 39.5 ppm, respectively. The following NMR experiments were carried out:

### A. ¹H-NMR:

pulse program:zg30 1D ¹H experiment with 30 degree pulse
   B. 2D: ¹H,¹H-COSY:
pulse program:cosygpmfphpp phase sensitive experiment with double quantum filter (Derome and Williamson 1990)
   C. 2D: ¹H, ¹H-ROESY:
pulse program: h-roesy_2.3_pp phase sensitive experiment with 180x/180-x spin-lock including a purge pulse (Bax and Davis (1985), Hwang and Shaka (1992))
   D. 2D: single bond ¹H,¹³C correlation (¹³C-DEPT-HSQC):
pulse program:hsqcedetgpsisp2.2 HSQC experiment with multiplicity editing, ¹H detection and z-gradient (Kay, Keifer and Saarinen 1992)
   E. 2D: long range ¹H,¹³C correlation (¹³C-HMBC):
pulse program: hmbcgplpndqf HMBC experiment with ¹H detection and z-gradient (Bax and Summers 1986)

### LC/MS Experimental Conditions:

Mass spectra were acquired on LC-MS systems using electrospray, Mass Spectrometer [M+H]+ refers to protonated molecular ion of the chemical species.

### Method 1:

### Pumps: Shimadzu Nexera LC-30AD

Mobile phase: A: water+0.05% formic acid+3.75 mMol ammonium acetate
B: acetonotrile+0.1% formic acid

Gradient:

| time (min) | % A | % B |
|---|---|---|
| 0 | 95 | 5 |
| 8 | 0 | 100 |
| 10 | 0 | 100 |
| 10.1 | 95 | 5 |
| 14 | 95 | 5 |

Flow rate: 100 μl/min
Split: no split

### Autosampler: Shimadzu Nexera SIL-30AC

Sample volume: <1 μl
Temperature: 15° C.
   Column oven: Shimadzu Nexera CTO-30A
Column1: Atlantis dC18, 3 μm, 1.0×150 mm

### or Column2: Acquity UPLC BEH C18, 1.7 μm, 1.0×50 mm

Temperature: 50° C.
   Diode array detector: Shimadzu Prominence SPD-M20A
Wavelength range: 200 500 nm
Sampling frequency: 6.25 Hz
Wave step: 2 nm
Time Constant: 0.64 sec
Slit Width: 1.2
Temperature 40° C.
   Software: Shimadzu instrument driver 5.5

### Mass Spectrometry (MS):

### Instrument: LTQ Orbitrap XL

### Software: Xcalibur 2.1.0 SP1

LTQ Orbitrap XL 2.5.5 5P2
   Ionization: electrospray
   Polarity: positive ions
   Spray voltage: 4.5 kV
   Capillary voltage: 32 V
   Capillary temperature: 250° C.
   Tube lens: 95V

### Analyzer: FTMS

### Resolution: 30000

Mass range: m/z 100-2000
Data type: profile

### Method 2:

### Waters Acquity UPLC/QT

### Pump Waters Acquity UPLC BSM (Binary Solvent Manager)

### Sampler Waters Acquity UPLC SO (SampleOrganizer)

Waters Acquity UPLC SM (Sample Manager)
   Column oven Waters Acquity UPLC CM (Column Manager)

### Detector Waters Acquity UPLC PDA (Photo Diode Array)

### MS Waters Acquity QT (Time Of Flight)

Eluent A Water+0.05% Formic acid+3.75 mM Ammonium acetate

### Eluent B Acetonitrile+0.04% Formic Acid

### Column Waters Acquity HSS T3 1.8 μm 2.1×50 mm

Column temperature 80° C.
Injection-Vol. 1 μl, partial loop
PDA Full scan 210-400 nm and one user selectable wavelength
Flow 1.0 ml/min
Stop Time 5.00 min

Gradient

| Time | % A (Eluent A) | % B (Eluent B) |
|---|---|---|
| 0.00 | 95 | 5 |
| 4.40 | 2 | 98 |
| 4.80 | 2 | 98 |
| 4.90 | 95 | 5 |
| 5.00 | 95 | 5 |

Mass range ESI+/−: 50-2500 m/z

### Example 1

### ¹H-NMR

1H NMR (600 MHz, DMSO-d6) δ ppm 1.18-1.30 (m, 2H) 1.72 (d, J=11.71 Hz, 2H) 1.82-2.00 (m, 2H) 2.13 (t, J=7.70 Hz, 2H) 2.85-3.07 (m, 2H) 3.68-3.78 (m, 1H) 3.88 (d, J=11.34 Hz, 2H) 4.68 (t, J=6.40 Hz, 1H) 5.06 (s, 2H) 7.41 (d, J=1.83 Hz, 2H) 7.57 (s, 1H) 7.66 (br. s., 1H) 7.77 (d, J=7.32 Hz, 1H)

### LC/MS:

Method 1: Rt=6.05 min; MS m/z [M+H]⁺ 456.1
Method 2: Rt=1.95 min; MS m/z [M-FI-1]⁺ 456.1

### Example 2

### ¹H-NMR

1H NMR (600 MHz, DMSO-d6) δ ppm 1.22-1.33 (m, 2H) 1.70-1.79 (m, 2H) 2.48 (t, J=6.77 Hz, 2H) 2.86-3.09 (m, 2H) 3.22 (t, J=6.77 Hz, 2H) 3.68-3.78 (m, 1H) 3.90 (d, J=11.34 Hz, 2H) 5.08 (s, 2H) 7.43 (d, J=1.83 Hz, 2H) 7.58 (d, J=1.46 Hz, 1H) 7.89 (d, J=7.68 Hz, 1H) 8.52 (br. s., 1H)

### LC/MS:

Method 1: Rt=5.81 min; MS m/z [M-FI-1]⁺456.1
Method 2: Rt=1.80 min; MS m/z [M+H]⁺ 456.1

### Biological Data:

The compounds of the invention are suitable as ATX inhibitors and may be tested in the following assays.

Reagents—LPC (oleoyl (18:1)) was purchased from Avert Polar Lipids (Alabaster, Ala.) and solubilized in methanol to 20 mM. Amplex Red was obtained from Invitrogen Life Technologies (Paisley, UK) and dissolved in DMSO to 10 mM. Choline oxidase and horseradish peroxidase (HRP) were obtained from Sigma Aldrich (Dorset, UK) and dissolved in HBSS to 20 U/ml and 200 U/ml respectively. All reagents were stored at −20° C. in single use aliquots. All experimental measurements were performed in assay buffer made up immediately prior to use (HBSS, 0.01% BSA essentially fatty acid free).

Protein—Recombinant human ATX was prepared at Novartis (Basel, CH) in a human embryonic kidney (HEK) cell preparation, and stored in single use aliquots of 26 mg/ml (26 μM) stocks stored at −80° C.

Method—All experimental measurements were performed in black 384 well polystyrene (low volume, round bottom, Corning (3676)) plates. PerkinElmer EnVision (Fluorescence Intensity/Absorbance Monochromator) or Tecan Infinite 200 PRO series plate reader was used to detect change in fluorescent intensity.

Assessing ATX inhibition—ATX activity was determined by measurement of released choline in reactions containing ATX (10 nM), choline oxidase (0.1 U/ml), HRP (100 U/ml), amplex red (50 μM) and LPC 18:1 (10 μM). Compounds of the invention were prepared as 10 point serial dilutions from 1 μM in duplicate and pre-incubated with ATX at 37° C. for 20 minutes prior to the addition of remaining reagents. The liberated choline was measured from changes in fluorescence intensity (Aex 530 nm, Aem 590 nm) of the product resurofin at 37° C. every 2 minutes over a 40-minute period. ATX activity was measured as a slope of the linear portion of the progress curve, typically between 14 to 24 minutes.

Data analysis—Slope data was exported to Graphpad prism (Graphpad software, San Diego, Calif.) where data was fitted to equation 1.

Y=Bottom+(Top−Bottom)/(1+10̂((Log IC50−X)*HillSlope)) Equation 1:

IC₅₀ values are determined from the concentration of compound that reduced the total activity by 50% and represent the mean of n≧2.

Table 1: The following table gives the IC₅₀ values for the exemplified compounds as measured in the above assay

TABLE 1

| Example | | IC₅₀ |
|---|---|---|
| no. | Compound | (μM) |
| 1 | 3,5-dichlorobenzyl 4-(4-oxo-4-(1H-1,2,3-triazol-4- | 0.07 |
| | yl)butanamido)piperidine-1-carboxylate | |
| 2 | 3,5-dichlorobenzyl 4-(4-hydroxy-4-(1H-1,2,3-triazol-4- | 0.01 |
| | yl)butanamido)piperidine-1-carboxylate | |

1. A compound that is chosen from 3,5-dichlorobenzyl 4-(4-oxo-4-(1H-1,2,3-triazol-4-yl)butanamido)piperidine-1-carboxylate and
3,5-dichlorobenzyl 4-(4-hydroxy-4-(1H-1,2,3-triazol-4-yl)butanamido)piperidine-1-carboxylate; or a pharmaceutically acceptable salt thereof.

2. A compound according to claim 1 that is 3,5-dichlorobenzyl 4-(4-oxo-4-(1H-1,2,3-triazol-4-yl)butanamido)piperidine-1-carboxylate or a pharmaceutically acceptable salt thereof.

3. A compound according to claim 1 that is 3,5-dichlorobenzyl 4-(4-hydroxy-4-(1H-1,2,3-triazol-4-yl)butanamido)piperidine-1-carboxylate or a pharmaceutically acceptable salt thereof.

4. A pharmaceutical composition comprising a compound according to claim 2, or a pharmaceutically acceptable salt thereof, and a pharmaceutically acceptable carrier.

5. A pharmaceutical combination comprising a therapeutically effective amount of the compound according to claim 2, or a pharmaceutically acceptable salt thereof, and one or more therapeutically active co-agent.

6-9. (canceled)

10. A method of treating an autotaxin dependent or an autotaxin mediated disease or condition selected from fibrosis, pruritus, cirrhosis, cancer, diabetes, kidney diseases, pain, asthma and COPD comprising administering to the subject a therapeutically effective amount of a compound according to claim 2.

11-12. (canceled)

13. The method according to claim 10, wherein the disease or condition is idiopathic pulmonary fibrosis.

14. A pharmaceutical composition comprising a compound according to claim 3, or a pharmaceutically acceptable salt thereof, and a pharmaceutically acceptable carrier.

15. A pharmaceutical combination comprising a therapeutically effective amount of the compound according to claim 3, or a pharmaceutically acceptable salt thereof, and one or more therapeutically active co-agent.

16. A method of treating an autotaxin dependent or an autotaxin mediated disease or condition selected from fibrosis, pruritus, cirrhosis, cancer, diabetes, kidney diseases, pain, asthma and COPD comprising administering to the subject a therapeutically effective amount of a compound according to claim 3.

17. The method according to claim 16, wherein the disease or condition is idiopathic pulmonary fibrosis.
